(19) United States
(12) Patent Application Publication
(10) Pub. No.: US 2017/0029453 A1
(43) Pub. Date: Feb. 2, 2017

(54) BIS(PHOSPHINE)-CARBODICARBENE CATALYST COMPLEXES AND METHODS OF USING THE SAME

(71) Applicant: The University of North Carolina at Chapel Hill, Chapel Hill, NC (US)

(72) Inventors: Simon Meek, Durham, NC (US); Courtney Roberts, Durham, NC (US); Matthew Goldfogel, Carrboro, NC (US); Matthew Joannou, Chapel Hill, NC (US)

(73) Assignee: The University of North Carolina at Chapel Hill, Chapel Hill, NC (US)

(21) Appl. No.: 15/302,699

(22) PCT Filed: Apr. 14, 2015
(86) PCT No.: PCT/IB2015/052721

§ 371 Date: Oct. 7, 2016

Related U.S. Application Data
(60) Provisional application No. 61/979,749, filed on Apr. 15, 2014.

(51) Int. Cl. C07F 15/00; B01J 31/22; C07F 9/6561; C07D 295/145; C07C 213/08; C07D 295/03; C07C 227/08; C07D 207/46; B01J 31/24; C07C 209/02

(52) U.S. Cl. CPC C07F 15/0073; B01J 31/2452; B01J 31/2273; C07F 9/6561; C07C 209/02; C07C 213/08; C07D 295/03; C07C 227/08; C07D 207/46; C07D 295/145; B01J 2231/321; B01J 2531/822; C07C 2101/14; C07C 2101/16

(57) ABSTRACT

An organometallic complex of a tridentate bis(phosphine)-carbodicarbene ligand and a transition metal, is described. In some embodiments the ligand has the structure of Formula (I): The complexes are useful in methods of making an allylic amine carried out by reacting a 1,3-diene with a substituted amine in the presence of such an organometallic complex to produce by intermolecular hydroamination the allylic amine.

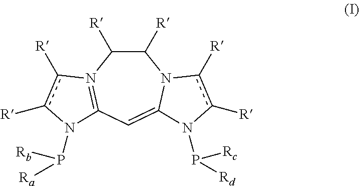

### RELATED APPLICATIONS

This application claims the benefit of U.S. Provisional Patent Application Ser. No. 61/979,749, filed Apr. 15, 2014, the disclosure of which is incorporated by reference herein in its entirety.

### FIELD OF THE INVENTION

The present invention concerns carbodicarbene ligands, complexes thereof, and methods of using the same for the intermolecular hydroamination of 1,3-dienes.

### BACKGROUND OF THE INVENTION

Carbon-based donors represent an important class of ligands for transition metals that promote multiple reaction types.¹ A significant objective in developing catalytic reactions is the design and synthesis of new classes of ligands. Accordingly, the development of new classes of carbon-based ligands for use in transition metal catalysis is an important goal in chemical synthesis. Carbodicarbenes (CDCs),^{2,3} also referred to as bent-allenes, are a family of compounds that contain a divalent carbon(0) center, captodatively stabilized by two carbene donors. These ligands can effectively bind to transition metals, and their σ- and π-electron donating properties have been established both experimentally⁴ and by theoretical calculations⁵ to be stronger than those of N-heterocyclic carbenes (NHCs). Surprisingly, only monodentate carbodicarbene complexes have been characterized (Au (1),^{4d,6} Ru (2),⁷ Fe,⁸ Rh,^{4a-c,9} Pd⁹) to date, in addition, there is an absence of reports demonstrating the ability of CDCs to act as effective ligands for transition metal catalysis.

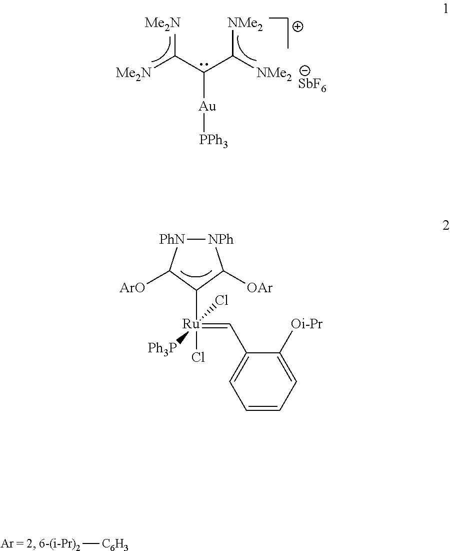

Furthermore, metal complexes supported by tridentate CDC-based ligands have not been prepared despite the utility of pincer scaffolds in promoting a number of important reactions.¹⁰

### SUMMARY OF THE INVENTION

In light of these above limitations, we initiated a program for the study of a new class of tridentate bis(phosphine)-carbodicarbenes and examined their ability to yield catalysis and effect a number of useful transformations.

Herein we report the synthesis, structure, and catalytic activity of easily prepared tridentate bis(phosphine)-(CDC)-Rh(I) complexes that effect formation of allylic amines via selective intermolecular hydroamination of 1,3-dienes with aryl and alkyl amines. Development of general catalytic procedures for the synthesis of functionalized unsaturated N containing molecules by the direct addition of amines to C—C π-bonds is a desirable, atom-economical transformation for chemical synthesis.¹¹ Transition metal-catalyzed intermolecular addition of amines to dienes to selectively afford allylic amines has been studied;^{12,13} however, poor control of site selectivity and the lack of a general catalytic system capable of both aryl and alkyl amine additions limits the field.14 Catalytic protocols have focused on the use of aryl and alkyl amines in order to obtain high site-selectivity.¹² The (CDC)-Rh(I) promoted hydroaminations described herein proceed with low catalyst loadings (1-5 mol %) and are tolerant of both alkyl and aryl amines; levels of site-selectivity and efficiency are complementary to previous intermolecular metal-catalyzed methods. Notably, the identity of the phosphine substituents (aryl vs. alkyl) plays an important role in determining the catalyst activity.

A first aspect of the present invention is an organometallic complex comprising: (a) a tridentate bis(phosphine)-carbodicarbene ligand, and (b) a transition metal.

A second aspect of the invention is a reaction mixture comprising an organometallic complex as described herein (e.g., as a catalyst), a solvent, a 1-3, diene substrate, and a substituted amine substrate.

A third aspect of the invention is a method of making an allylic amine, comprising reacting a 1,3-diene with a substituted amine in the presence of an organometallic complex of claim 1-7 in a catalytic amount

A fourth aspect of the invention is a tridentate bis(phosphine)-carbodicarbene ligand as described herein.

The foregoing and other objects and aspects of the present invention are explained in greater detail in the specification set forth below.

### DETAILED DESCRIPTION OF THE PREFERRED EMBODIMENTS

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all United States patents cited herein are to be incorporated herein by reference in their entirety.

“Alkyl” as used herein alone or as part of another group, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Loweralkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. Such groups can be unsubstituted or substituted with one or more (e.g., one, two, three four, etc.) independently selected electron-donating or electron-withdrawing groups

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or loweralkyl. The term “cycloalkyl” is generic and intended to include heterocyclic groups as discussed below unless specified otherwise. Such groups can be unsubstituted or substituted with one or more (e.g., one, two, three four, etc.) independently selected electron-donating or electron-withdrawing groups.

“Alkenyl” as used herein alone or as part of another group, refers to a straight or branched chain hydrocarbon containing from 1 to 10 carbon atoms (or in loweralkenyl 1 to 4 carbon atoms) which include 1 to 4 double bonds in the normal chain. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. Such groups can be unsubstituted or substituted with one or more (e.g., one, two, three four, etc.) independently selected electron-donating or electron-withdrawing groups.

“Alkynyl” as used herein alone or as part of another group, refers to a straight or branched chain hydrocarbon containing from 1 to 10 carbon atoms (or in loweralkynyl 1 to 4 carbon atoms) which include 1 triple bond in the normal chain. Representative examples of alkynyl include, but are not limited to, 2-propynyl, 3-butynyl, 2-butynyl, 4-pentynyl, 3-pentynyl, and the like. The term “alkynyl” or “loweralkynyl” is intended to include both substituted and unsubstituted alkynyl or loweralkynyl unless otherwise indicated and these groups may be substituted with the same groups as set forth in connection with alkyl and loweralkyl above. Such groups can be unsubstituted or substituted with one or more (e.g., one, two, three four, etc.) independently selected electron-donating or electron-withdrawing groups.

“Heterocyclo” as used herein alone or as part of another group, refers to an aliphatic (e.g., fully or partially saturated heterocyclo) or aromatic (e.g., heteroaryl) monocyclic- or a bicyclic-ring system. Monocyclic ring systems are exemplified by any 3 to 8 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic and heterocyclic ring systems fused to an aryl group as defined herein, a cycloalkyl group as defined herein, or another monocyclic or heterocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. Such groups can be unsubstituted or substituted with one or more (e.g., one, two, three four, etc.) independently selected electron-donating or electron-withdrawing groups.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include heteroaryl, and both substituted and unsubstituted aryl/heteroaryl unless otherwise indicated and these groups may be substituted with the same groups as set forth in connection with alkyl and loweralkyl above. Such groups can be unsubstituted or substituted with one or more (e.g., one, two, three four, etc.) independently selected electron-donating or electron-withdrawing groups.

“Heteroaryl” as used herein is as described in connection with heterocyclo above. Such groups can be unsubstituted or substituted with one or more (e.g., one, two, three four, etc.) independently selected electron-donating or electron-withdrawing groups.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Heteroarylalkyl” as used herein alone or as part of another group, refers to a heteroaryl group, as defined herein, appended to the parent molecular moiety through an alkyl group, as defined herein.

“Heterocycloalkyl” as used herein alone or as part of another group, refers to a heterocyclo group, as defined herein, appended to the parent molecular moiety through an alkyl group, as defined herein.

“Electron-withdrawing group” and “electron donating group” refer to groups having the ability of a substituent to withdraw or donate electrons relative to that of hydrogen if the hydrogen atom that occupied the same position in the molecule is replaced therewith. These terms are well understood by one skilled in the art and are discussed in Advanced Organic Chemistry, by J. March, John Wiley and Sons, New York, N.Y., pp. 16-18 (1985), incorporated herein by reference. Examples of such electron withdrawing and electron donating groups or substituents include, but are not limited to halo, nitro, cyano, carboxy, loweralkenyl, loweralkynyl, loweralkanoyl (e.g., formyl), carboxyamido, aryl, quaternary ammonium, aryl (loweralkanoyl), carbalkoxy and the like; acyl, carboxy, alkanoyloxy, aryloxy, alkoxysulfonyl, aryloxysulfonyl, and the like; hydroxy, alkoxy or loweralkoxy (including methoxy, ethoxy and the like); loweralkyl; amino, lower alkylamino, di(loweralkyl) amino, aryloxy (such as phenoxy), mercapto, loweralkylthio, lower alkylmercapto, disulfide (loweralkyldithio) and the like; 1-piperidino, 1-piperazino, 1-pyrrolidino, acylamino, hydroxyl, thiolo, alkylthio, arylthio, aryloxy, alkyl, ester groups (e.g., alkylcarboxy, arylcarboxy, heterocyclocarboxy), azido, isothiocyanato, isocyanato, thiocyanato, cyanato, and the like. One skilled in the art will appreciate that the aforesaid substituents may have electron donating or electron withdrawing properties under different chemical conditions. Moreover, the present invention contemplates any combination of substituents selected from the above-identified groups. See U.S. Pat. Nos. 6,133,261 and 5,654,301; see also U.S. Pat. No. 4,711,532.

“Acyl” as used herein alone or as part of another group refers to a —C(O)R radical, where R is any suitable substituent such as aryl, alkyl, alkenyl, alkynyl, cycloalkyl or other suitable substituent as described herein.

“Alkanoyl” refers to the group —C(O)R′, wherein R′ is lower alkyl. Hence “alkanoyl” groups are particular examples of “acyl” groups, as described above.

“Halo” or “halogen,” as used herein refers to —Cl, —Br, —I or —F.

“Oxy” as used herein refers to an —O— group.

“Sulfonyl,” as used herein, refers to an —SO₂— group.

“Thio” as used herein refers to an —S— group.

“Hydrocarbyl” as used herein may be any suitable aromatic, aliphatic, or mixed aromatic/aliphatic group, for example containing from 1 to 30 carbon atoms or more, and optionally containing heteroatoms. Examples of hydrocarbyl groups include but are not limited to alkyl, cycloalkyl, alkenyl, alkynyl, aryl, heterocyclo, heteroaryl, arylalkyl, heteroarylalkyl, heterocycloalkyl, arylalkyloxy, arylalkylamino, arylalkylthio, heteroarylalkyloxy, heteroarylalkylamino, heteroarylalkylthio, heterocycloalkyloxy, heterocycloalkylamino, heterocycloalkylthio, arylaminooxy, heteroarylaminooxy, heterocycloaminooxy, aryloxyamino, heteroaryloxyamino, heterocyclooxyamino, arylalkyl, heteroarylalkyl, heterocycloalkyl, arylalkenyl, heteroarylalkenyl, heterocycloalkenyl, arylalkynyl, heteroarylalkynyl, heterocycloalkynyl, arylhydrazino, heteroarylhydrazino, heterocyclohydrazino, arylazo, heteroarylazo, heterocycloazo, arylalkylaminoalkyl, heteroarylalkylaminoalkyl, heterocycloalkylaminoalkyl, arylalkyloxyalkyl, heteroarylalkyloxyalkyl, heterocycloalkyloxyalkyl, each of which can be unsubstituted or substituted with one or more (e.g., one, two, three, four) independently selected electron-donating or electron-withdrawing groups.

“Linking group” as used herein may be any suitable linking group, including aromatic, aliphatic and mixed aromatic and aliphatic linking group.

“Solid support” as used herein may be porous or nonporous, in any suitable form, and formed from any suitable material such as alumina, silica, titania, kieselguhr, diatomaceous earth, bentonite, clay, zirconia, magnesia, zeolites, carbon black, activated carbon, graphite, fluoridated carbon, organic polymers, metals, metal alloys, and combinations thereof, in accordance with known techniques. See, e.g., U.S. Pat. No. 6,908,873.

### 1. Ligands and Organometallic Complexes.

As noted above, the present invention provides a tridentate bis(phosphine)-carbodicarbene ligand (i.e., pincer ligands), and complexes thereof with a transition metal. The complexes are useful as catalysts as described further below.

In some embodiments, the ligand has the structure of Formula I:

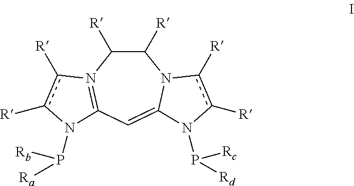

wherein:

each dashed line independently represents an optional double bond;

Rₐ, R_{b}, R_{c}, and R_{d} are each independently selected alkyl, aryl, arylalkyl, alkoxy, amino, or substituted amino;

each R′ is an independently selected hydrogen, hydrocarbyl group, electron donating group, or electron-withdrawing group;

or at least one R′ is S-L-, where S is a solid support and L is a linking group.

Particular examples of the foregoing include but are not limited to the structures of Formula Ia, Formula Ib, and Formula Ic:

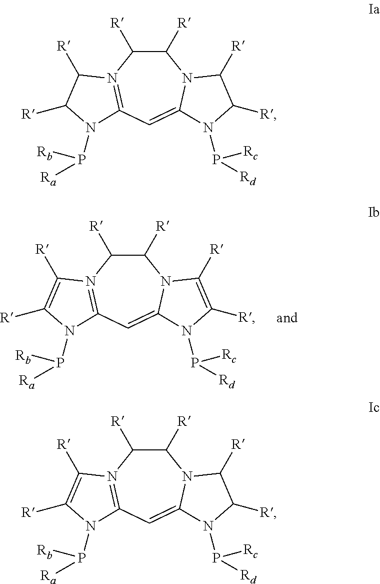

where the substituents are as given above.

Any suitable transition metal may be used in the complexes described herein, including but not limited to ruthenium, nickel, palladium, platinum, rhodium, iridium, cobalt, iron, silver, gold, and molybdenum.

In some embodiments, Rₐ, R_{b}, R_{c}, and R_{d} are each independently selected alkyl or aryl.

In some embodiments, each R′ is hydrogen, or independently hydrogen, halo, loweralkyl, loweralkoxy, or hydroxy.

The complexes may be immobilized covalently or noncovalently on a solid support. Thus in some embodiments at least one R′ may be S-L-, where S is a solid support and L is a linking group.

In some embodiments catalysts as described above have the structure of Formula I′:

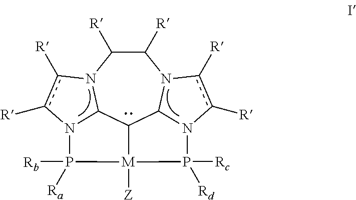

wherein:

each dashed line independently represents an optional double bond, substituents are as given above, M is said transition metal; and Z is halo.

Complexes can be immobilized as catalysts can be immobilized by covalent coupling to a grafted or functionalized polystyrene support (e.g. Wang resin, Argogel resin, Merrifield resin, Tentagel resin, etc.) in accordance with known techniques. See, e.g., U.S. Pat. No. 6,951,958. Catalysts can be immobilized by covalent coupling through a silicon or siloxane containing-linker to any suitable solid support, such as silica, in accordance with known techniques. Catalysts can be immobilized by Lewis acid:Lewis base interactions, or binding to any suitable solid support, such as alumina, without covalent coupling, in accordance with known techniques.

### 2. Catalytic Methods.

A reaction mixture can be prepared comprising an organometallic complex as described above, a solvent, a 1-3, diene substrate, and a substituted amine substrate. Solvents and reaction conditions are not critical and any suitable reaction system can be employed.

As noted above, the invention provides a method of making an allylic amine, comprising: reacting a 1,3-diene with a substituted amine in the presence of an organometallic complex as described above (e.g., in a catalytic amount) to produce by intermolecular hydroamination said allylic amine.

In some embodiments, the allylic amine has the structure of Formula II:

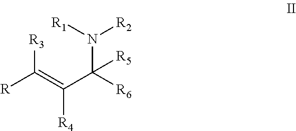

wherein:

R, R₁, and R₂ are independently selected hydrocarbyl groups;

R₃, R₄ and R₅ are independently selected hydrogen or hydrocarbyl groups; and

R₆ is alkyl (e.g., methyl) or arylalkyl (e.g., benzyl).

In some embodiments, the 1,3-diene has the structure of Formula III:

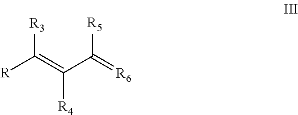

wherein:

R is hydrocarbyl;

R₃, R₄ and R₅ are independently selected H or hydrocarbyl; and

R₆ is alkyl (e.g., CH₂) or arylalkyl (e.g., benzyl);

or a pair of R₃, R₄, and R₅ optionally form a linking group (e.g., a C2, C3, or C4 alkylene).

In some embodiments, the substituted amine has the structure of Formula IV:

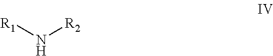

wherein R₁ and R₂ are independently selected hydrocarbyl groups.

The present invention is explained in greater detail in the following non-limiting Examples.

### EXPERIMENTAL

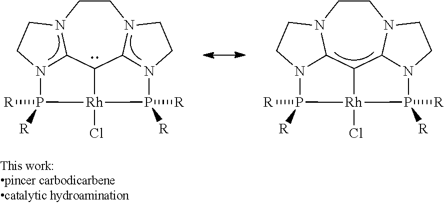

We initiated our studies by the synthesis of the required 1,4-diazepenium salts. As shown in Scheme 1, phosphination of heterocyclic base 3 with Ph2PCl (or i-Pr2PCl) in the presence of Et3N affords 1,4-diazepenium salts 4 and 5 in 85% and 71% yield. Both salts are bench stable and purified by silica gel column chromatography. The tetrafluoroborate salts 4 and 5 may then undergo cyclometallation by treatment with a suspension of [Rh(cod)Cl]2 in THF at 22° C., followed by deprotonation of the corresponding cationic Rh(II)-H with NaOMe (THF, 22° C.) to afford square-planar (CDC)-Rh(I) complexes 6 and 7 as orange/yellow solids in 98% yield.¹⁵

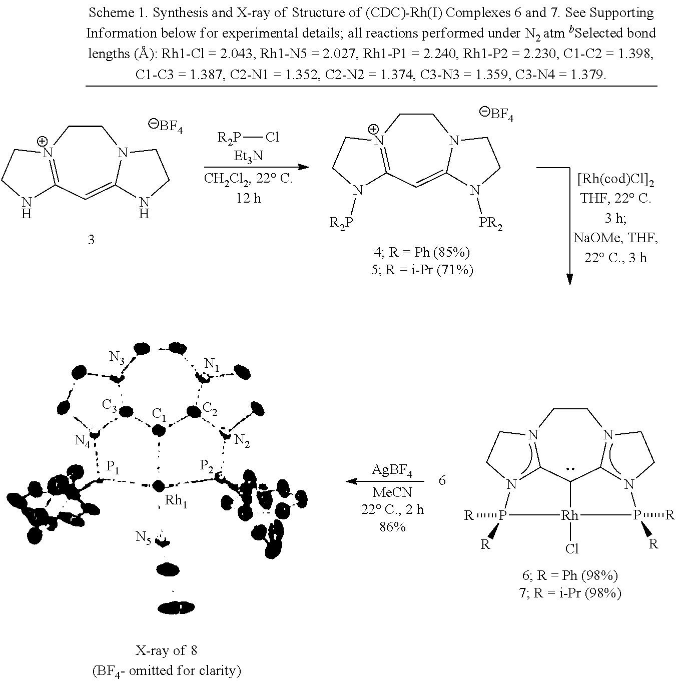

The 13C NMR signal of the carbodicarbene carbon(0) is indicated by a doublet-of-triplets in the 13C{1H} NMR spectrum; 72.98 ppm for 6 (1JRh=36.0 Hz, 1JP=11.7 Hz), and 73.74 ppm for 7 (1JRh=36.3 Hz, 1JP=10.4 Hz). These values are consistent with those previously reported by Bertrand and Fürstner with the upfield shift indicating the electron rich nature of the divalent carbon(0).^{4,6} To elucidate some of the structural features of (CDC)-Rh complexes (Scheme 1), we obtained the X-ray crystal structure of acetonitrile complex 8.¹⁶ As indicated by the ORTEP diagram, the Rh1-C1 bond length is 2.043 Å. Bond lengths of the CDC ligand indicate a carbodicarbene structure with the average C3→C1 bond lengths of 1.395 Å in comparison to shorter N2-C2 carbene of the NHCs (average 1.365 Å). The Rh1-N5 bond length of 2.029 Å indicates the strong trans influence of the carbodicarbene carbon.¹⁷

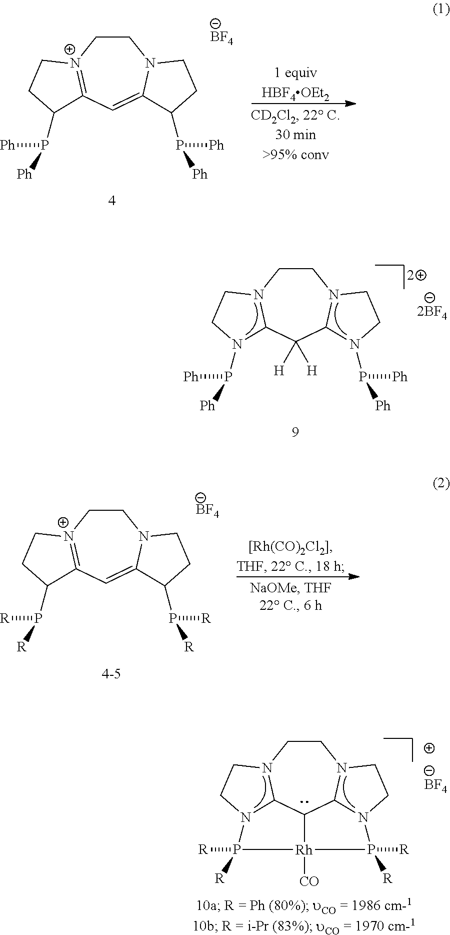

To gain insight into the electronic nature of the ligand, 4 was treated with one equivalent HBF4-OEt₂ in CD₂Cl₂ at 22° C. (Eq 1), which generated dication 9. The symmetrical 1H NMR confirms protonation at the central carbon in accord with previously described systems.5e,6b This demonstrates the presence of significant electron density at the central carbon of cation 4 and supports its reactivity as a carbodicarbene. Further measure of the electron donating properties of the CDCs derived from 4 and 5, was evaluated through the carbonyl stretching frequencies of 10a-b (Eq 2). The cationic Rh(I) complexes exhibit infrared vco values (10a, 1986 cm-1; 10b, 1970 cm-1) lower than those observed for analogous cationic Rh(I) pincer complexes.18

With complexes in hand we began to investigate whether Rh(I) complexes 6 and 7 are effective catalysts for hydroamination. As the data in Table 1 illustrate, the ability of Rh(I) complexes 6 and 7 to catalyze the hydroamination of phenyl 1,3-butadiene with aniline requires an additive; <2% conv is observed entries 1 and 2. In contrast, as shown in entries 3 and 4, when 5 mol % (CDC)-Rh and 5 mol % AgBF₄ are used (80° C., C6H5Cl), the reactions proceed to deliver allylic amine 11 (>98% Markovnikov site-selectivity) in 66% and 65% isolated yield, respectively. Less coordinating ions (PF6, SbF6, and OTf) are less efficient (39-59% yield; entries 5-7). Gratifyingly, the hydroamination can be effected with 1 mol % 6 to deliver 11 (63% conversion) in slightly diminished yield (59%). Catalytic hydroamination with 5 mol % Rh(I)-NCMe complex 8 (entry 9), affords 11 in similar conversion (72%) compared to catalysts generated in situ with silver(I) salts, suggesting that a cationic Rh(I) complex is the active catalyst. Control reactions with HBF₄—OEt₂ and AgBF4 (entries 10 and 11) exclude an acid- or silver(I)-catalyzed process.

TABLE 1
Evaluation of (CDC)-Rh(I) Complexes in Hydroamination^{a}

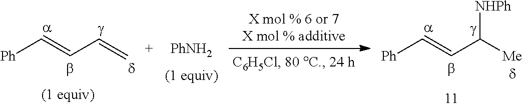

| entry | complex; mol % | additive; mol % | conv (%)^{b} | yield (%)^{c} |
|---|---|---|---|---|
| 1 | 6; 5 | — | <2 | nd |
| 2 | 7; 5 | — | <2 | nd |
| 3 | 6; 5 | AgBF₄; 5 | 75 | 66 |
| 4 | 7; 5 | AgBF₄; 5 | 73 | 65 |
| 5 | 6; 5 | AgPF₆; 5 | 70 | 59 |
| 6 | 6; 5 | AgSbF₆; 5 | 40 | 31 |
| 7 | 6; 5 | AgOTf; 5 | 60 | 51 |
| 8 | 6; 1 | AgBF₄; 1 | 63 | 59 |
| 9 | 8; 5 | — | 72 | 67 |
| 10 | — | HBF₄·OEt₂; 5 | <2 | nd |
| 11 | — | AgBF₄; 5 | <2 | nd |

TABLE 2
(CDC)-Rh-Catalyzed Hydroaminations of Phenyl-1,3-Butadiene with Aryl
and Secondary Alkyl Amines^{a}

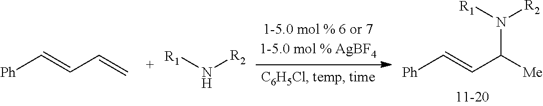

| | | complex; | | | | |
|---|---|---|---|---|---|---|
| entry | amine; product | mol % | temp (° C.) | time (h) | conv (%)^{b} | yield (%)^{c} |
| 1 | C₆H₅NH₂; 11 | 6; 1 | 60 | 24 | 88 | 71 |
| 2 | p-CF₃C₆H₄NH₂; 12 | 7; 2 | 60 | 24 | 96 | 91 |
| 3 | p-MeOC₆H₄NH₂; 13 | 7; 3 | 60 | 48 | 68 | 64 |
| 4 | o-BrC₆H₄NH₂; 14 | 8; 3 | 50 | 48 | 86 | 85 |
| 5 | o-MeC₆H₄—NH₂; 15 | 7; 5 | 60 | 48 | 89 | 80 |
| 6 | morpholine; 16 | 7; 3 | 80 | 48 | 92 | 89 |
| 7 | pyrrolidine; 17 | 6; 5 | 80 | 48 | 80 | 75^{d} |
| 8 | Bn₂NH; 18 | 7; 2 | 80 | 48 | 58 | 56 |
| 9 | Bn(Me)NH; 19 | 7; 5 | 80 | 48 | 74 | 72 |
| 10 | n-Pr₂NH; 20 | 7; 5 | 80 | 48 | 14^{e} | 6 |

^{a-c} See Table 1. ^{d}With 20 mol % NH₄BF₄ additive; 11% without NH₄BF₄. ^{e}12% conv at 100° C.

Next, we examined the influence of changing the identity of the amine on the activity of (CDC)-Rh(I)-catalyzed hydroamination. As the representative examples in Table 2 demonstrate, Rh-complexes 6 and 7 catalyzed hydroamination of phenyl 1,3-butadiene with various aryl and alkyl amines to generate allylic amines in >98% γ-selectivity. The findings in entries 2 and 3 of Table 2 illustrate that allylic aryl amines with electron-withdrawing (12) and electron-donating (13) groups can be accessed with high site-selectivity; the reaction of p-CF3-substituted aniline proves to be slightly more efficient. Sterically hindered o-bromoaniline and o-toluidine (entries 4 and 5) require 3-5 mol % of 6 and 7 to generate allylic amines 14 and 15 with complete site-selectivity in 85% and 80% yield, respectively. As shown in entries 6 and 7, cyclic alkyl amines morpholine and pyrrolidine are tolerated and react to furnish allylic amines 16 (89% yield) and 17 (75% yield); however, pyrrolidine requires the use of 20 mol % NH₄BF₄ additive. Moreover, secondary alkyl amines bearing benzyl (entries 8 and 9) and n-propyl (entry 10) groups can participate in Rh-catalyzed site-selective hydroamination albeit with varying efficiency. Two points regarding Table 2 merit mention. First, the optimal complex (6 vs. 7) and reaction conditions in each case vary depending on the amine structure.19 Second, in general, (CDC)-Rh-catalyzed hydroaminations with alkyl amines require higher temperatures (80° C.) to proceed compared to aryl amines (50-60° C.).

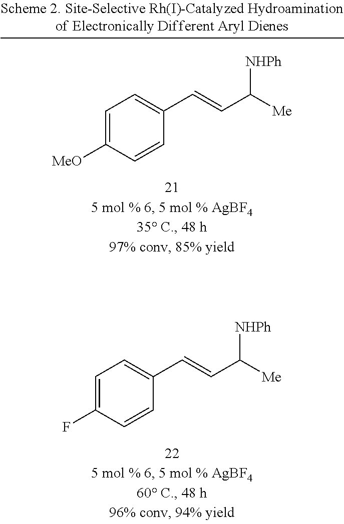

To further evaluate the catalytic properties of (CDC)-Rh(I), we investigated the reaction with respect to the electronics of the aryl diene component. A notable aspect of these studies is the observed increased reactivity of complex 6 with electronically disparate dienes. As illustrated in Scheme 2, Rh catalyzed addition of aniline to p-MeO-substituted diene occurs at significantly lower temperature compared to p-F-substituted (35° C. versus 60° C.) to afford 20 and 21 in >85% yield.

TABLE 3
(CDC)-Rh(I)-Catalyzed Hydroaminations of Dienes with Aniline^{a}

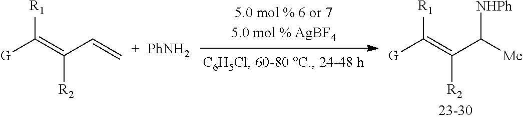

| | | complex; | | |
|---|---|---|---|---|
| entry | diene | temp (° C.) | product | yield (%)^{b} |
| 1 | 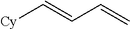 | 6; 60 | 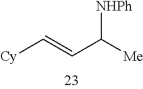 | 89 |
| 2 | 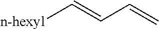 | 6; 70 | 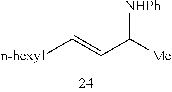 | 70^{c} |
| 3 | 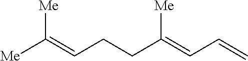 | 6; 60 | 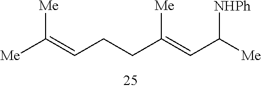 | 97 |
| 4 | 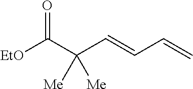 | 6; 80 | 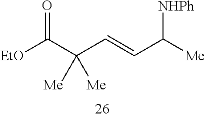 | 78 |
| 5 | 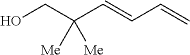 | 6; 80 | 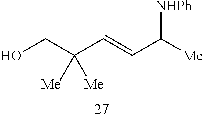 | 74 |
| 6 | 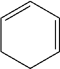 | 6; 60 | 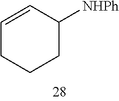 | 96^{d} |
| 7 | 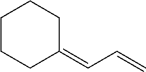 | 6; 60 | 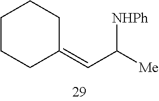 | 77 |
| 8 | 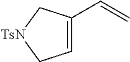 [SMILES: C=CC1=CCN(S(=O)(=O)c2ccc(C)cc2)C1] | 7; 65 | 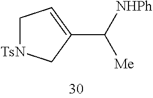 | 69 |

^{a}See Supporting Information for experimental details; all reactions performed under N2 atm with 2 equiv. diene; up to >98% site-selectivity.
^{b}Yields of purified products are an average of two runs.
^{c}3:2 mixture of γ:α addition
^{d}4 equivalents of diene were used.

Rhodium-catalyzed diene hydroaminations promoted by pincer carbodicarbene complexes display significant synthetic scope. As the representative examples in Table 3 indicate, Rh complexes 6 and 7 promote the hydroamination of alkyl diene substrates to deliver allylic amine products bearing di- or trisubstituted olefins (up to >98% γ-selectivity). Under optimal reaction conditions (5 mol % 6 at 60° C.) cyclohexyl butadiene is efficiently converted to 23 in 89% yield (entry 1). It is worthy of note that n-alkyl derived substrates undergo efficient catalytic hydroamination to generate allylic amines but as mixtures of constitutional isomers; 24 (5 mol % 6, 70° C.; entry 2) is generated in 70% as an inseparable 3:2 mixture of γ:α addition products. This underscores a current limitation of Rh complexes 6 and 7 towards site-selective hydroamination of sterically unbiased 1,3-dienes. As illustrated in entries 3, 7, and 8, trisubstituted 1,3-dienes undergo site-selective (>98%) Rh-catalyzed hydroamination (5 mol % 6 or 7, 80° C., 48 h) to deliver the corresponding allylic amines in good yield: 25 (97%), 29 (77%), and 30 (69%). The Rh-catalyzed protocol is also effective for the generation of cyclic allylic amines as demonstrated by the formation 28 (entry 6) in 96% yield. It should be noted that a number of functional groups are compatible under the relatively mild reaction conditions, including: alkenes (entry 3), esters (entry 4), alcohols (entry 5), and N-tosyl amines (entry 8).

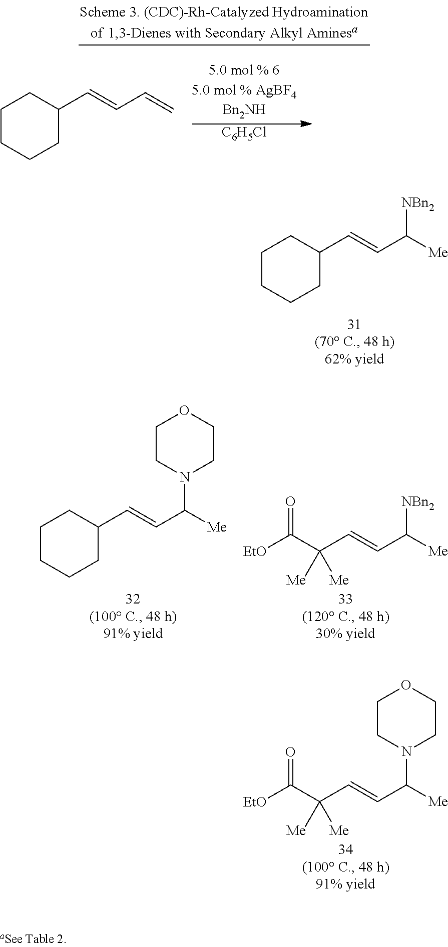

The four representative examples in Scheme 3 further underline the generality and synthetic utility of this (CDC)-Rh(I) hydroamination protocol. As noted (vide supra), catalytic hydroamination with aliphatic amines generally requires higher temperatures (70-120° C.) versus aryl amines. Siteselective formation of aliphatic allylic amines 31 (62%) and 32 (91%) from dibenzyl amine and morpholine proceeds efficiently in the presence of 5 mol % 6 (70 and 100° C.). Incorporation of ester functionality is also tolerated as catalytic hydroamination (5 mol % (CDC)-Rh 6, and 5 mol % AgBF₄) delivers 33 (120° C., 48 h) and 34 (100° C., 48 h) in modest to excellent yields (30% and 91%). In conclusion, we have developed a tridentate carbodicarbene ligand scaffold that enables efficient Rh-catalyzed site selective intermolecular hydroamination of 1,3-dienes compatible with both alkyl and aryl amines. The reactions described, represent the first examples of a carbodicarbene transition metal complex that functions as an effective catalyst.²⁰

### REFERENCES

(1) Díez-González, S.; Marion, N.; Nolan, S. P. Chem. Rev. 2009, 109, 3612-3676.
(2) Frenking, G.; Tonner, R. Carbodicarbenes, In Contemporary Carbene Chemistry (Eds: Moss, R. A.; Doyle, M. P.), John Wiley & Sons, Inc, Hoboken, N.J., 2013; Ch 8, pp. 216.
(3) Kaufhold, O.; Hahn, F. E. Angew. Chem. Int. Ed. 2008, 47, 4057-4061. (b) Melaimi, M.; Soleilhavoup, M.; Bertrand, G. Angew. Chem. Int. Ed. 2010, 49, 8810-8849.
(4) (a) Dyker, C. A.; Lavallo, V.; Donnadieu, B.; Bertrand, G. Angew. Chem. Int. Ed. 2008, 47, 3206-3209. (b) Lavallo, V.; Dyker, C. A.; Donnadieu, B.; Bertrand, G. Angew. Chem. Int. Ed. 2008, 47, 5411-5414. (c) Melaimi, M.; Parameswaran, P.; Donnadieu, B.; Frenking, G.; Bertrand, G. Angew. Chem. Int. Ed. 2009, 48, 4792-4795. (d) Alcarazo, M.; Lehmann, C. W.; Anoop, A.; Thiel, W.; Fürstner, A. Nature Chem. 2009, 1, 295-301.
(5) (a) Tonner, R.; Frenking, G. Angew. Chem. Int. Ed. 2007, 46, 8695-8698. (b) Tonner, R.; Frenking, G. Chem. Eur. J. 2008, 14, 3260-3272. (c) Tonner, R.; Frenking, G. Chem. Eur. J. 2008, 14, 3273-3289. (d) Tonner, R.; Heydenrych, G.; Frenking, G. Chem-PhysChem 2008, 9, 1474-1481. (e) Fernádez, I.; Dyker, C. A.; DeHope, A.; Donnadieu, B.; Frenking, G.; Bertrand, G. J. Am. Chem. Soc. 2009, 131, 11875-11881. (f) Klein, S.; Tonner, R.; Frenking, G. Chem. Eur. J. 2010, 16, 10160-10170.
(6) Fürstner, A.; Alcarazo, M.; Goddard, R.; Lehmann, C. W. Angew. Chem. Int. Ed. 2008, 47, 3210-3214.
(7) DeHope, A.; Donnadieu, B.; Bertrand, G. J. Organomet. Chem. 2011, 696, 2899-2903.
(8) Pranckevicius, C.; Stephan, D. W. Organometalllics 2013, 32, 2693-2697.
(9) Chen, W.-C.; Hsu, Y.-C.; Lee, C.-Y.; Yap, G. P. A.; Ong, T.-G. Organometallics 2013, 32, 2435-2442.
(10) (a) The Chemistry of Pincer Compounds (Eds: Morales-Morales, D.; Jensen, C.), Elsevier: Amsterdam, 2007. (b) Organometallic Pincer Chemistry (Eds: van Koten, G.; Milstein, D.), Top. Organomet. Chem., 2013, 40. (c) Albrecht, M.; van Koten, G. Angew. Chem. Int. Ed. Engl. 2001, 40, 3750-3781. (d) Selander, N.; J Szabó, K. Chem. Rev. 2011, 111, 2048-2076. (e) Choi, J.; MacArthur, A. H. R.; Brookhart, M.; Goldman, A. S. Chem. Rev. 2011, 111, 1761-1779.
(11) (a) Müller, T. E.; Beller, M. Chem. Rev. 1998, 98, 675-704. (b) Roesky, P. W.; Miller, T. E. Angew. Chem. Int. Ed. 2003, 42, 2708-2710. (c) Hong, S.; Marks, T. J. Acc. Chem. Res. 2004, 37, 673-686. (d) Miller, T. E.; Hultzsch, K. C.; Yus, M.; Foubelo, F.; Tada, M. Chem. Rev. 2008, 108, 3795-3892.
(12) For Pd-catalyzed examples, see: (a) Löber, O.; Kawatsura, M.; Hartwig, J. F. J. Am. Chem. Soc. 2001, 123, 4366-4367. (b) Minami, T.; Okamoto, H.; Ikeda, S.; Tanaka, R.; Ozawa, F.; Yoshifuji, M. Angew. Chem. Int. Ed. 2001, 40, 4501-4503. (c) Johns, A. M.; Utsunomiya, M.; Incarvito, C. D.; Hartwig, J. F. J. Am. Chem. Soc. 2006, 128, 1828-1839. (d) Kuchenbeiser, G.; Shaffer, A. R.; Zingales, N. C.; Beck, J. F.; Schmidt, J. A. R. J. Organomet Chem. 2011, 696, 179-187. For a Ni-catalyzed example, see: (e) Pawlas, J.; Nakao, Y.; Kawatsura, M.; Hartwig, J. F. J. Am. Chem. Soc. 2002, 124, 3669-3679. For a Ru-catalyzed example, see: (f) Yi, C. S.; Yun, S. Y. Org. Lett. 2005, 7, 2181-2183. For Ca- and Sr-catalyzed examples, see: (g) Brinkmann, C.; Barrett, A. G. M.; Hill, M. S.; Procopiou, P. A. J. Am. Chem. Soc. 2012, 134, 2193-2207. For a Ti-catalyzed example, see: (h) Preuβ, T.; Saak, W.; Doye, S. Chem. Eur. J. 2013, 19, 3833-3837.
(13) For related catalytic intermolecular hydroamidations of 1,3-dienes, see: (a) Brouwer, C.; He, C. Angew. Chem. Int. Ed. 2006, 45, 1744-1747. (b) Giner, X.; Nájera, C. Org. Lett. 2008, 10, 2919-2922. (c) Qin, H.; Yamagiwa, N.; Matsunaga, S.; Shibasaki, M. J. Am. Chem. Soc. 2006, 128, 1611-1614. (d) Giner, X.; Nájera, C.; Kovács, G.; Lledós, A.; Ujaque, G. Adv. Synth. Catal. 2011, 353, 3451-3466. (e) Banerjee, D.; Junge, K.; Beller, M. A. Angew. Chem. Int. Ed. 2014, 53, 1630-1635.
(14) For examples of catalytic intramolecular hydroamination and hydroamidation of 1,3-dienes, see: (a) Hong, S.; Marks, T. J. J. Am. Chem. Soc. 2002, 124, 7886-7887. (b) Hong, S.; Kawaoka, A. M.; Marks, T. J. J. Am. Chem. Soc. 2003, 125, 15878-15892. (c) Shapiro, N. D.; Rauniyar, V.; Hamilton, G. L.; Wu, J.; Toste, F. D. Nature 2011, 470, 245-249. (d) Kanno, O.; Kuriyama, W.; Wang, Z. J.; Toste, F. D. Angew. Chem. Int. Ed. 2011, 50, 9919-9922. (e) Deschamp, J.; Collin, J.; Hannedouche, J.; Schulz, E. Eur. J. Org. Chem. 2011, 3329-3338. (f) Pierson, J. M.; Ingalls, E. L.; Vo, R. D.; Michael, F. E. Angew. Chem. Int. Ed. 2013, 52, 13311-13313.
(15) Attempts to deprotonate 4 and 5, and isolate the free CDC were unsuccessful.
(16) Complexes 6 and 7 crystallize as plates and X-ray quality crystals could not be obtained at the present time.
(17) For a cationic PNP-Rh(I)-NCMe complex, see: (a) Hahn, C.; Sieler, J.; Taube, R. Polyhedron 1998, 17, 1183-1193. (b) Hermann, D.; Gandelman, M.; Rozenberg, H.; Shimon, L. J.; Milstein, D. Organometallics 2002, 21, 812-818. For a POP-Rh(I)-NCMe complex, see: (c) Julian, L. D.; Hartwig, J. F. J. Am. Chem. Soc. 2010, 132, 13813-13822.
(18) (a) Feller, M.; Ben-Ari, E.; Gupta, T.; Shimon, L. J. W.; Leitus, G.; Diskin-Posner, Y.; Weiner, L.; Milstein, D. Inorg. Chem. 2007, 46, 10479-10490. (b) Feller, M.; Diskin-Posner, Y.; Shimon, L. J. W.; Ben-Ari, E.; Milstein, D. Organometallics 2012, 31, 4083-4101. (c) See reference 17 (c).
(19) Please see Supporting Information.

### Supporting Information

General:

All reactions were carried out in flame or oven (140° C.) dried glassware that had been cooled under vacuum. Unless otherwise stated, all reactions were carried out under an inert N₂ atmosphere. All reagents were purged or sparged with N₂ for 20 min prior to distillation or use. All solid reagents were dried by azeotropic distillation with benzene three times prior to use. Infrared (IR) spectra were obtained using a Jasco 460 Plus Fourier transform infrared spectrometer or a ASI ReactIR 1000, Model: 001-1002 for air sensitive rhodium carbonyl complexes. Mass spectra were obtained using a Micromass Quattro-II triple quadrupole mass spectrometer in combination with an Advion NanoMate chip-based electrospray sample introduction system and nozzle for low-res or Waters Q-ToF Ultima Tandem Quadrupole/Time-of-Flight Instrument UE521 at University of Illinois at Urbana Champaign for high-res or Waters Q-ToF Xevo Tandem Quadrupole/Time-of-Flight Instrument. The Q-Tof Ultima mass spectrometer was purchased in part with a grant from the National Science Foundation, Division of Biological Infrastructure (DBI-0100085). All samples were prepared in MeOH or MeCN for metal complexes. Proton and carbon magnetic resonance spectra (1H NMR and 13C NMR) were recorded on a Bruker model DRX 400 or a Bruker AVANCE III 600 CryoProbe (1H NMR at 400 MHz or 600 MHz, 13C NMR at 100 or 150 MHz, 31P NMR at 160 or 243 MHz and 19F NMR at 376 or 564 MHz) spectrometer with solvent resonance as the internal standard (1H NMR: CDCl₃ at 7.26 ppm, CD₂Cl₂ at 5.30 ppm, C₆D₆ at 7.16 ppm, CD₃CN at 1.94 ppm; 13C NMR: CDCl₃ at 77.16 ppm, C₆D₆ at 128.4 ppm, CD3CN at 1.31 ppm). NMR data are reported as follows: chemical shift, integration, multiplicity (s=singlet, d=doublet, t=triplet, dd=doublet of doublets, td=triplet of doublets, dt=doublet of triplets, ddd=doublet of doublet of doublets, septetd=septet of doublets, m=multiplet, bs=broad singlet, bm=broad multiplet), and coupling constants (Hz). X-ray diffraction studies were conducted on a Bruker-AXS SMART APEXII diffractometer. Crystals were selected and mounted using Paratone oil on a MiteGen Mylar tip.

(E)-phenyl-1,3-butadiene,1

(E)-1-(buta-1,3-diene-1-yl)-4-methoxybenzene,2 (E)-1-(buta-1,3-diene-1-yl)-4-fluorobenzene,1,3 (E)/(Z)-1-buta-1,3-dien-1-ylcylohexane,2 (E)-deca-1,3-diene,4(E)-4,8-dimethylnona-1,3,7-triene,5 (E)-ethyl-2,2-dimethylhexa-3,5-dienoate,6,7 (E)-2,2-dimethylhexa-3,5-dien-1-ol,⁸ allylidenecyclohexane⁹ were synthesized according to a literature method or a modified literature method and matched reported spectra.

Solvents:

Solvents were purged with argon and purified under a positive pressure of dry argon by a SG Waters purification system: dichloromethane (EMD Millipore) and THF (EMD Millipore) were passed through activated alumina columns. Chlorobenzene (Alfa Aesar) was dried over K₂CO₃, distilled under vacuum and stored over activated 5 Å molecular sieves in a dry box.

Reagents:

Acetonitrile—d3 was purchased from Cambridge Isotope Labs, dried over CaH2 and stored in a dry box over activated 4 Å molecular sieves. AgNO₃ Doped Silica Gel was prepared as a 1% mixture by weight as described in the literature.10 Aniline was purchased from Aldrich, dried on CaH2, distilled under vacuum, and stored in a dry box freezer at −30° C. p-Anisidine was purchased from Alfa Aesar, dried over CaCl₂, distilled under vacuum, and stored in a dry box. Benzylmethylamine was purchased from Alfa Aesar, dried over K₂CO₃, distilled under vacuum, and stored in a dry box. 2-Bromoaniline was purchased from Alfa Aesar, dried over CaCl₂, distilled under vacuum, and stored in a dry box. Chlorobenzene was dried over K₂CO₃, distilled under vacuum and stored over activated 5 Å molecular sieves in a dry box. Chloroform-dl was purchased from Cambridge Isotope Labs, dried over CaH₂ and stored in a dry box over activated 4 Å molecular sieves. Chloro(1,5-cyclooctadiene)rhodium(I) dimer was purchased from Pressure Chemicals, stored in a dry box and used as received. Chlorodiisopropyl phosphine was purchased from Acros Organics and used as received. Chlorodiphenylphosphine was purchased from Alfa Aesar and used as received. Cyclohexa-1,3-diene was purchased from Alfa Aesar and was distilled and stored under N2 at −20° C. Dichloromethane—d2 was purchased from Cambridge Isotope Labs, dried over CaH₂ and stored in a dry box over activated 4 Å molecular sieves. Dibenzyl amine was purchased from Alfa Aesar, passed through a plug of alumina onto activated 5 Å molecular sieves for 24 h and transferred to a vial in a dry box. Di-n-propyl amine was purchased from Aldrich, dried over KOH, and distilled under reduced pressure and stored in a dry box. Morpholine was purchased from Alfa Aesar, dried over KOH, distilled under reduced pressure and stored in a dry box. Pyrrolidine was purchased from Alfa Aesar, dried over Na, distilled under reduced pressure and stored in a dry box. Silver tetrafluoroborate was purchased from Strem, stored in a dry box, and used without further purification. 4-(Trifluoromethyl)aniline was purchased from Alfa Aesar, distilled over CaH₂, and stored in at −30° C. in a dry box freezer. Sodium methoxide was purchased from Strem, stored in a dry box, and used as received. o-Toluidine was purchased from Alfa Aesar, dried over CaH2, distilled under vacuum, and stored in a dry box. Tetrafluoroboric acid was purchased from Alfa Aesar and used as received. Triethylamine was purchased from Fisher and dried over CaH₂ and distilled immediately prior to use.

Procedures for Preparation of CDC 1,4-Diazapenium Salts 4 and 5:

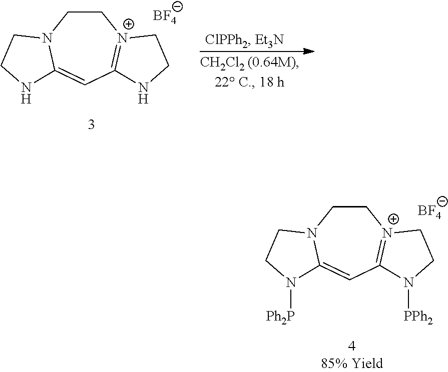

### Synthesis of pH(CDC)-BF4 Salt 4

A 250 mL round-bottom flask with a stir bar was charged with diazepinium salt 311 (2.00 g, 7.52 mmol), sealed with a septum and purged with nitrogen. Dichloromethane (12.0 mL, [ ]=0.640 M) and triethylamine (12.0 mL, 860 mmol) were added via syringe and the solution was allowed to stir for 5 min. To the yellow heterogenous solution, chlorodiphenylphosphine (4.05 mL, 22.6 mmol) was added via syringe and the reaction was allowed to stir at 22° C. for 18 h. The reaction was triturated with diethyl ether (100 mL) and filtered to isolate a yellow solid. The yellow solid was purified by SiO₂ column chromatography (20:1 to 9:1 CH₂Cl₂/MeOH). After concentrating the solution to a solid, the resulting yellow residue was dissolved in benzene (5 mL) before being triturated with toluene (150 mL) to produce a white powder which was filtered off (4.10 g, 6.39 mmol 85% yield). Excess water was removed by azeotropic distillation with benzene (3×3 mL).

¹H NMR (600 MHz, CDCl₃): δ 7.41 (20H, m), 6.17 (1H, t, J=7.3 Hz), 3.80 (4H, s), 3.77 (4H, t, J=8.8 Hz), 3.34 (4H, t, J=8.8 Hz). 13C NMR (150 MHz, CDCl₃): δ 163.90 (d, J=19.2 Hz), 132.98 (d, J=9.2 Hz), 132.50 (d, J=17.0 Hz), 130.43, 129.18 (d, J=5.4 Hz), 62.21 (t, J=26.4 Hz), 50.92, 47.52, 44.40 (d, J=7.3 Hz). 31P NMR (243 MHz, CDCl3): δ 41.4. 19F NMR (376 MHz): δ −153.33 (d, J=19.8 Hz). IR (ν/cm-1): 3057 (w), 2891 (w), 1594 (w), 1557 (s), 1524 (s), 1508 (w), 1478 (m), 1436 (m), 1312 (w), 1292 (m), 1227 (m), 1161 (w), 1097 (w), 1054 (s). HRMS (ES+) [M+H]+ calcd for C₃₃H₃₃N₄P₂⁺ 547.2175. found: 547.2172.

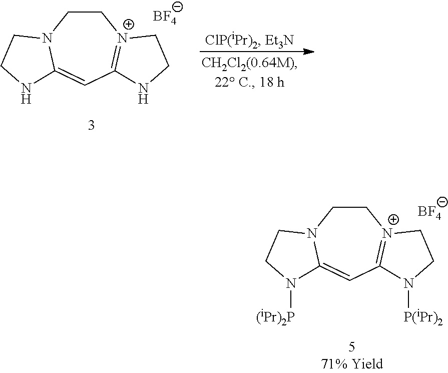

Synthesis of iPr(CDC)-BF4 Salt 5.

A 250 mL round-bottom flask with a stir bar was charged with diazepinium salt 311 (2.00 g, 7.52 mmol), sealed with a septum and purged with nitrogen. Dichloromethane (12.0 mL, [ ]=0.640 M) and triethylamine (12.0 mL, 860 mmol) were added via syringe and the solution was allowed to stir for 5 min. To the yellow heterogeneous solution, chlorodiisopropylphosphine (3.6 mL, 22.6 mmol) was added via syringe and the reaction was allowed to stir at 22° C. for 18 h. The reaction was filtered through a pad of CELITEe® media, which was washed with dichloromethane (100 mL). The filtrate was concentrated and purified by SiO₂ column chromatography (30:1 CH₂Cl₂/MeOH). The resulting off-white solid was dissolved in a minimal amount of dichloromethane and triturated with hexanes to produce a white powdery solid which was filtered off (2.65 g, 71% yield). Excess water was removed by azeotropic distillation with benzene (3×3 mL).

¹H NMR (400 MHz, CDCl3): δ 5.70 (1H, t, J=7.0 Hz), 3.73 (4H, m), 3.65, (4H, s), 3.59 (4H, m), 2.08 (4H, septd, J=7.0, 1.6 Hz), 1.11 (12H, dd, J=16.8, 7.0 Hz), 1.03 (12H, dd, J=12.5, 7.0 Hz). 13C NMR (100 MHz, CDCl₃): δ 165.04 (d, J=21.3 Hz), 63.63 (t, J=29.7 Hz), 51.13, 47.27, 44.55, 25.11 (d, J=15.1 Hz), 19.21 (d, J=19.21 Hz), 18.80 (d, J=22.9 Hz). 31P NMR (162 MHz, CDCl₃): δ 63.24. 19F NMR (376 MHz): δ −153.64 (d, J=18.8 Hz). IR (ν/cm-1): 2952 (m), 2924 (w), 2867 (m), 1557 (s), 1523 (m), 1507 (w), 1457 (w), 1436 (w), 1386 (m), 1291 (m), 1227 (m), 1163 (w), 1053 (s). HRMS (ES+) [M+H]+ calcd for C₂₁H₄₁N4P₂^{+×}411.2801. found: 411.2799.

General Procedure for the Preparation of (CDC)-Rh(I)Cl Complexes 6 and 7:

In an N₂ filled dry box, a 20-mL scintillation vial with a stir bar was charged with (CDC)-BF4 salt (1.0 equiv) and chloro(1,5-cyclooctadiene)rhodium(I) dimer (0.50 equiv). Tetrahydrofuran was added, the vial capped, and the resulting mixture was allowed to stir for 3 h at 22° C. The solution was concentrated in vacuo. Residual 1,5-cyclooctadiene was removed by azeotropic distillation with benzene (3×1 mL). Sodium methoxide (1.0 equiv) and THF were added to the reaction vial. The resulting heterogeneous mixture was allowed to stir for 3 h at 22° C.

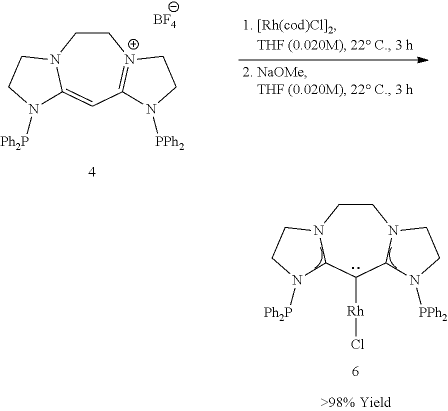

Synthesis of pH(CDC)RhCl Complex 6.

Following the general procedure for the preparation of (CDC)-Rh(I)Cl complexes, ^{Ph}(CDC)-BF₄ 4 (258 mg, 0.406 mmol) and chloro(1,5-cyclooctadiene)rhodium(I) dimer (100 mg, 0.203 mmol) were solvated with THF (10 mL, [ ]=0.020 M). After concentration, NaOMe (21.9 mg, 0.406 mmol) was added and solvated with THF (10 mL, [ ]=0.020 M). After reaction was complete, acetonitrile (4.0 mL) was added to the solution, which was then filtered through a pad of Celite® media. The Celite® pad was washed with a 1:1 mixture of THF:MeCN (5 mL) to dissolve the solid. The resulting filtrate was concentrated to afford orange solid 6 in >98% yield (282 mg, 0.412 mmol).

¹H NMR (600 MHz, CD₃CN): δ 7.65-7.68 (8H, m), 7.50-7.56 (12H, m), 4.01 (4H, t, J=8.1 Hz), 3.45 (4H, s), 3.30 (4H, t, J=8.3 Hz). 13C NMR (150 MHz, CD₃CN): δ 173.11 (td, J=18.1, 1.3 Hz), 134.61, (t, J=17.0 Hz), 133.15 (t, J=6.8 Hz), 131.70, 129.82 (t, J=3.8 Hz), 72.98 (dt, J=29.9, 9.7 Hz), 58.88, 47.32, 42.24. 31P NMR (243 MHz, CD3CN): δ 79.20 (d, J=170.4 Hz). HRMS (ES+) [M−Cl]+ calcd for C33H32N4P2Rh+ 649.1157. found: 649.1155. When formic acid was added to neutral complex 6 the protonated N2 adduct was formed. HRMS (ES+) [M+H+N2]+ calcd for C₃₃H₃₃N₆P₂RhCl+ 713.0985. found: 713.1317.

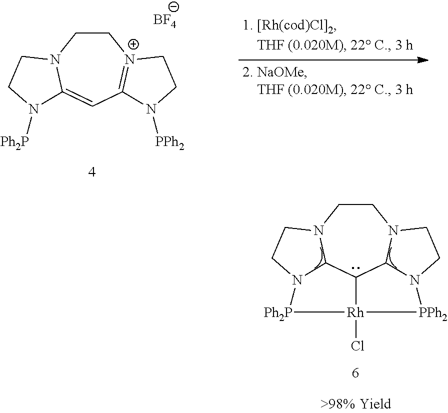

Synthesis of iPr(CDC)RhCl Complex 7.

Following the general procedure for the preparation of (CDC)-Rh(I)Cl complexes, iPr(CDC)BF₄ 5 (100 mg, 0.201 mmol) and chloro(1,5-cyclooctadiene)rhodium(I) dimer (49.5 mg, 0.100 mmol) were solvated with THF (5.0 mL, [ ]=0.020]. After concentration, NaOMe (10.9 mg, 0.201 mmol) was added and solvated with THF (5 mL, [ ]=0.020 M). The resulting yellow powder was filtered through a pad of Celite® and washed with THF (4×1 mL). The yellow solid was dissolved off the Celite® pad using acetonitrile (5 mL) and concentrated in vacuo to afford 7 in >98% yield (110 mg, 0.201 mmol) as a canary yellow powder.

¹H NMR (600 MHz, CD₃CN): δ 3.88 (4H, t, J=8.2 Hz), 3.42 (4H, t, J=8.2 Hz), 3.31 (4H, s), 2.29 (4H, septetd, J=7.0, 1.0 Hz), 1.27 (12H, m), 1.20 (12H, m). 13C NMR (150 MHz, CD₃CN): δ 173.95 (t, J=15.1 Hz), 73.76 (dt, J=30.1, 8.7 Hz), 58.69, 47.40, 42.66, 27.91 (t, J=8.1 Hz), 19.37, 18.89 (t, J=4.4 Hz). 31P NMR (162 MHz, CD3CN): 110.44 (d, J=167.1 Hz). HRMS (ES+) [M−Cl]+ calcd for C₂₁H₄₀N₄P2Rh+ 513.1783. found: 513.1795.

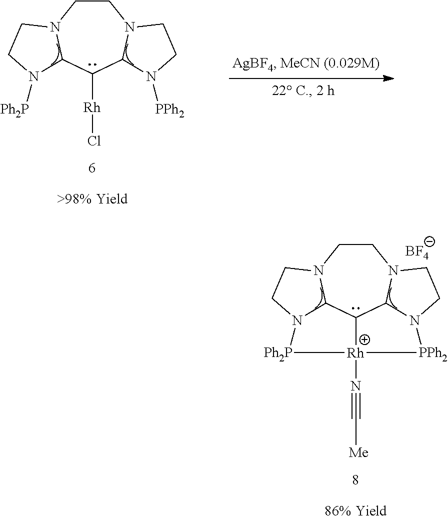

Synthesis of Cationic Ph(CDC)Rh-MeCN BF₄ Complex 8.

An 8-mL amber vial equipped with a stir bar was charged with 6 (40.0 mg, 0.0574 mmol) and AgBF4 (17.1 mg, 0.0876 mmol). Acetonitrile (2.0 mL, [ ]=0.029) was added to the vial and the heterogeneous solution was allowed to stir for 2 h at 22° C. The resulting solution was filtered through a pad of Celite® and concentrated to afford 8 (39.0 mg, 0.0502 mmol, 86% yield) as a dark orange powder. X-ray quality crystals of 8 were grown from a slow salt metathesis of 6 and NaBF4 in a 5:1 mixture of benzene:MeCN.

¹H NMR (600 MHz, CD=CN): δ 7.75-7.78 (12H, m), 7.68-7.70 (8H, m), 4.31 (4H, t, J=8.5 Hz), 3.78-3.80 (8H, m). 13C NMR (150 MHz, CD=CN): δ 168.67 (t, J=16.6 Hz), 134.75, 133.60 (t, J=7.1 Hz), 130.94 (t, J=5.8 Hz), 125.40, (t, J=29.8 Hz), 58.29, 56.33 (dt, J=28.1, 4.5 Hz), 47.51, 44.55. 31P NMR (162 MHz, CD3CN): 67.63 (d, J=58.4 Hz). 19F NMR (376 MHz): δ −152.24 (d, J=20.0 Hz). HRMS (ES+) [M+H]+ calcd for C═H═N═P=Rh+ 690.1423. found: 690.1435.

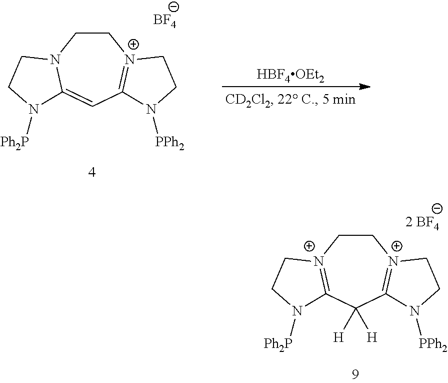

Synthesis of Dicationic ^{Ph}(CDC)-(BF₄)2 Salt 9.

In an N₂ filled dry box, an 8-mL vial was charged with 4 (10.0 mg, 0.016 mmol) and CD₂Cl₂ (0.25 mL). The solution was transferred to an NMR tube and the vial was washed with CD₂Cl₂ (2×0.25 mL). The tube was capped with a septum lined lid and brought outside the dry box. Tetrafluoroboric acid (5.1 μL, 0.019 mmol) was added via syringe which incited an immediate color change from pale yellow to almost colorless and the tube was shaken for 5 min before being analyzed.

¹H NMR (600 MHz, CD₂Cl₂): δ 7.44-7.7.52 (m, 20H), 5.36 (t, J=4.9 Hz), 4.05-4.09 (m, 8H), 3.64 (t, 4H, J=10.9 Hz.

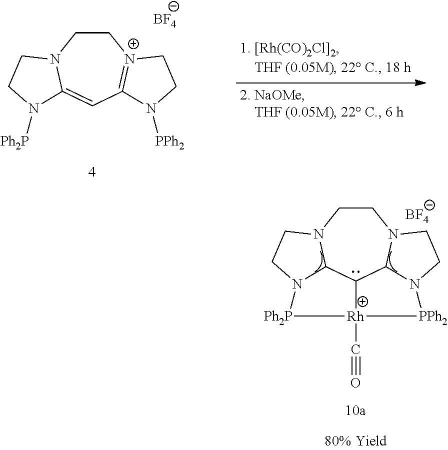

Synthesis of ^{Ph}(CDC)Rh—CO Complex 10a.

In an N₂ filled dry box, an 8-mL vial with a stir bar was charged with 4 (16.3 mg, 0.026 mmol) and dicarbonylchlororhodium(I) dimer (5.0 mg, 0.013 mmol), and tetrahydrofuran (0.50 mL, [ ]=0.050 M). The vial was capped and the resulting mixture was allowed to stir for 18 h at 22° C. The resulting solution was concentrated to afford a yellow powder. To this solid, NaOMe (1.4 mg, 0.026 mmol) was added followed by tetrahydrofuran (0.50 mL, [ ]=0.05 M). The yellow heterogeneous solution was allowed to stir for 6 h at 22° C. The solution was concentrated in vacuo, dissolved in CHCl₃ (1 mL), and filtered through a cotton plug which was washed with CHCl3 (2×1 mL). The filtrate was concentrated in vacuo to afford 10a in 80% yield (15.8 mg, 0.021 mmol) as a burnt yellow powder.

¹H NMR (600 MHz, CD₃CN): δ 7.67-7.70 (8H, m), 7.52-7.57 (4H, m), 7.50-7.54 (8H, m), 4.19 (4H, t, J=8.4 Hz), 3.65 (4H, s), 3.36 (4H, t, J=14.4 Hz). 13C NMR (150 MHz, CD₃CN): δ 194.6 (dt, J=57.2, 12.7 Hz), 174.4 (t, J=21.6 Hz), 133.4 (t, J=7.9 Hz), 132.9, 132.2, (t, J=27.1 Hz), 130.2, (t, J=5.4 Hz), 86.4 (dt, J=28.1, 11.0 Hz), 59.5, 47.2, 42.4. 31P NMR (162 MHz, CD3CN): δ 87.99 (d, J=103.6 Hz). IR (ν/cm-1) (CH₂Cl₂): 1986 (νCO), 1537 (m), 1475 (w), 1375 (w), 1267 (w). HRMS (ES+) [M−CO]+ calcd for C₃₄H₃₂N₄OP₂Rh+ 677.1101. found: 677.1809.

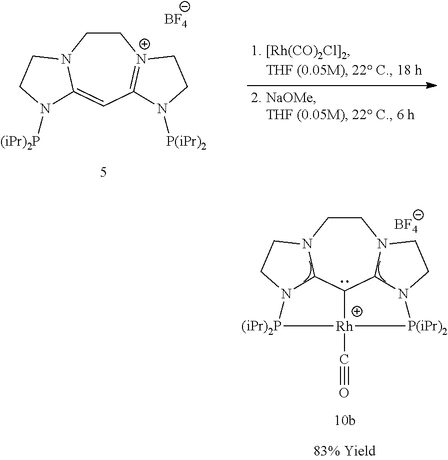

Synthesis of ^{iPr}(CDC)Rh—CO Complex 10b.

In an N2 filled dry box, an 8-mL vial with a stir bar was charged with 5 (13.0 mg, 0.026 mmol) and dicarbonylchlororhodium(I) dimer (5.0 mg, 0.013 mmol), and tetrahydrofuran (0.50 mL, [ ]=0.050 M). The vial was capped, and the resulting mixture was allowed to stir for 18 h at 22° C. The resulting solution was concentrated to afford a yellow powder. To this solid, NaOMe (1.4 mg, 0.026 mmol) was added followed by tetrahydrofuran (0.50 mL, [ ]=0.05 M). The yellow heterogeneous solution was allowed to stir for 6 h at 22° C. The solution was concentrated in vacuo to afford 10b in 83% yield (16.5 mg, 0.0216 mmol) as a canary yellow powder.

¹H NMR (600 MHz, CDCl₃): δ 4.11 (4H, t, J=8.52 Hz), 3.55 (4H, t, J=8.52 Hz), 3.53 (4H, s), 2.38 (4H, m), 1.28-1.32 (12H, m), 1.2-1.24 (12H, m). 13C NMR (150 MHz, CDCl3): δ 195.36 (dt, J=34.5, 19.0 Hz), 174.15 (t, J=19.1 Hz), 85.16 (dt, J=19.0, 9.3 Hz), 58.24, 46.37, 42.26, 27.31 (t, J=12.3 Hz), 18.95, 18.73 (t, J=4.3 Hz). 31P NMR (162 MHz, CDCl₃): δ 119.97 (d, J=98.8 Hz). IR (ν/cm-1) (CH₂Cl₂): 2966 (w), 2885 (w), 1970 (νCO), 1529 (m), 1475 (w), 1375 (w), 1182 (w), 1055 (s). HRMS (ES+) [M−CO]+ calcd for C₂₂H₄₀N₄OP2Rh+ 541.1727. found: 541.1715.

General Procedure for the (CDC)-Rh-Catalyzed Hydroaminations of Phenyl 1,3-Butadiene in Tables 1 and 2.

In an N2 filled dry box, an 8-mL vial equipped with a stir bar was charged with the appropriate (CDC)-RhCl complex and silver salt. Chlorobenzene was added via syringe, the vial was capped and the mixture allowed to stir for 1 h at 22° C. Reactions that did not require the addition of (CDC)-RhCl were also allowed to stir for 1 h at 22° C. for consistency. Aniline was added via syringe, followed by addition of the phenyl 1,3-butadiene. The vial was capped with a Teflon® lined lid or septum cap, taped with electrical tape and brought outside the dry box. Any volatile acids (HBF₄.OEt₂ and HCl-dioxane) were added via syringe through the Teflon® septa under an atmosphere of N₂. The reaction was allowed to warm to the appropriate temperature and stir for 24 h. The reaction was allowed to cool and an aliquot was taken to determine the conversion by ¹H NMR using DMF as an internal standard. The remaining solvent was removed in vacuo. The products were purified by SiO₂ column chromatography.

TABLE 1
Control reactions.

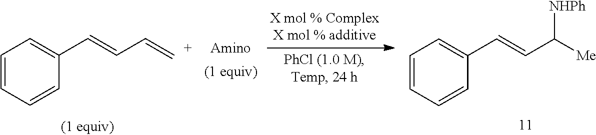

| | | | | | Conv. of | Conv. to | Isolated |
|---|---|---|---|---|---|---|---|
| Entry | Complex; mol % | Additive; mol % | Amine | Temp (° C.) | Diene (%) | Product (%) | Yield (%) |
| 1 | ^{Ph}PCP—Rh—Cl; 5 | — | aniline | 80 | 5 | <2 | — |
| 2 | ^{iPr}PCP—Rh—Cl; 5 | — | aniline | 80 | 4 | <2 | — |
| 3 | ^{Ph}PDP—Rh—Cl; 5 | AgBF₄; 5 | aniline | 80 | 85 | 75 | 67 |
| 4 | ^{iPr}PCP—Rh—C1; 5 | AgBF₄; 5 | aniline | 80 | 81 | 73 | 65 |
| 5 | ^{Ph}PCP—Rh—Cl; 5 | AgPF₆; 5 | aniline | 80 | 81 | 68 | 59 |
| 6 | ^{Ph}PCP—Rh—Cl; 5 | AgSbF₆; 5 | aniline | 80 | 91 | 40 | 31 |
| 7 | ^{Ph}PCP—Rh—C1; 5 | AgOTf; 5 | aniline | 80 | 87 | 60 | 51 |
| 8 | ^{Ph}PCP—Rh—Cl; 5 | AgBF₄; 5, | aniline | 80 | 96 | 30 | — |
| | | HBF₄; 20 | | | | | |
| 9 | ^{Ph}PCP—Rh—Cl; 1 | AgBF₄; 1 | aniline | 80 | 83 | 62 | 59 |
| 10 | — | HBF₄; 5 | aniline | 89 | 12 | <2 | — |
| 11 | — | HCl; 50 | aniline | 80 | 27 | <2 | — |
| 12 | — | HCl; 50 | aniline | 120 | 75 | <2 | — |
| 13 | — | AgBF₄; 5 | aniline | 80 | 12 | <2 | — |
| 14 | — | NH₄BF₄; 20 | aniline | 80 | 12 | <2 | — |
| 15 | — | NH₄BF₄; 20 | pyrrolidine | 80 | 31 | <2 | — |

All reactions were run according to the procedure outlined for Table 1.
1) The conversion to product is based off of an NMR conversion with an internal standard of DMF.

TABLE 2
Initial catalyst screen for (CDC)-Rh-Catalyzed Hydroaminations
of Phenyl-1,3-Butadiene with Aryl and Secondary Alkyl Amines^{a}

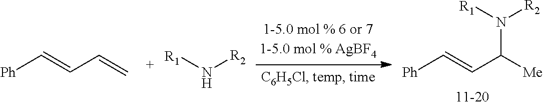

| | | complex; | | | |
|---|---|---|---|---|---|
| entry | amine; product | mol % | temp (° C.) | time (h) | conv (%)^{b} |
| 1 | C₆H₅NH₂; 11 | 6; 1 | 60 | 24 | 88 |
| 2 | C₆H₅NH₂; 11 | 7; 1 | 60 | 48 | 73 |
| 3 | p-CF₃C₆H₄NH₂; 12 | 6; 2 | 60 | 48 | 42 |
| 4 | p-CF₃C₆H₄NH₂; 12 | 7; 2 | 60 | 24 | 96 |
| 5 | p-MeOC₆H₄NH₂; 13 | 6; 2 | 60 | 48 | 38 |
| 6 | p-MeOC₆H₄NH₂; 13 | 7; 3 | 60 | 48 | 68 |
| 7 | o-BrC₆H₄NH₂; 14 | 6; 3 | 50 | 48 | 86 |
| 8 | o-BrC₆H₄NH₂; 14 | 7; 2 | 50 | 24 | 39 |
| 9 | o-MeC₆H₄—NH₂; 15 | 6; 5 | 60 | 48 | 55 |
| 10 | o-MeC₆H₄—NH₂; 15 | 7; 5 | 60 | 48 | 89 |
| 11 | morpholine; 16 | 6; 3 | 80 | 48 | 90 |
| 12 | morpholine; 16 | 7; 3 | 80 | 48 | 92 |
| 13 | pyrrolidine; 17 | 6; 5 | 80 | 48 | 80 |
| 14 | pyrrolidine; 17 | 7; 5 | 80 | 48 | <2 |
| 15 | Bn₂NH; 18 | 6; 2 | 90 | 48 | 10 |
| 16 | Bn₂NH; 18 | 7; 2 | 80 | 24 | 58 |
| 17 | Bn(Me)NH; 19 | 6; 5 | 80 | 36 | 61 |
| 18 | Bn(Me)NH; 19 | 7; 5 | 80 | 48 | 74 |
| 19 | n-Pr₂NH; 20 | 6; 5 | 80 | 48 | 14 |
| 20 | n-Pr₂NH; 20 | 7; 5 | 80 | 48 | 13 |

^{a}All reactions performed under N₂ atm; >98% site selectivity in all cases, see optimized reactions for concentrations and diene equivalents.
^{b}Values determined by analysis of 400 or 600 MHz ¹H NMR spectra of unpurified mixtures.

General Procedure for Rh-Catalyzed Hydroaminations in Scheme 2 and Tables 2, 3, and 4:

In an N₂ filled dry box, an 8-mL vial equipped with a stir bar was charged with (CDC)-RhCl, AgBF₄, and chlorobenzene. The vial was capped and the mixture allowed to stir at 22° C. for 1 h, to generate a heterogeneous, purple or blue solution. The appropriate amine was added via syringe (or weighed into the vial) followed by the 1,3-diene. The vial was capped with a Teflon® lined lid, sealed with electrical tape, brought outside the dry box, and heated to the indicated temperature for the appropriate amount of time. The reaction was allowed to cool to 22° C., and an aliquot was taken to determine the conversion by 1H NMR using an internal DMF standard. The remaining solvent was removed in vacuo. The products were purified by SiO₂ column chromatography to give isolated yields.

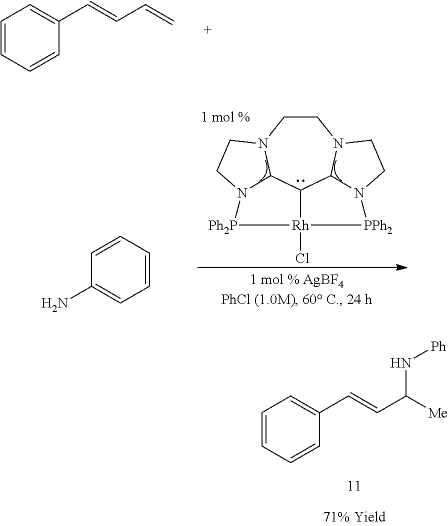

Synthesis of (E)-N-(4-phenylbut-3-en-2-yl)aniline 11 (Table 2, Entry 1).

Following the general procedure for (CDC)-Rh-catalyzed hydroamination, aniline (18.6 mg, 0.200 mmol) and phenyl 1,3-butadiene (26.0 mg, 0.200 mmol) were added to a solution of 6 (1.4 mg, 0.0020 mmol) and AgBF4 (0.4 mg, 0.0020 mmol) in chlorobenzene (200 μL, [ ]=1.00 M), and the reaction allowed to stir at 60° C. for 24 h. The resulting oil was purified by SiO2 column chromatography (20:1 Hex/Et₂O) to afford 11 (31.3 mg, 0.142 mmol, 71% yield) as a colorless oil.

¹H NMR (600 MHz, CDCl₃): δ 7.36 (2H, d, J=7.4 Hz), 7.30 (2H, t, J=7.6 Hz), 7.22 (1H, t, J=7.3 Hz), 7.17 (2H, t, J=8.0 Hz), 6.69 (1H t, J=7.3 Hz), 6.66 (2H, d, J=7.9 Hz), 6.58 (1H, d, J=16.0 Hz), 6.22 (1H, dd, J=15.9, 5.9 Hz), 4.14-4.17 (1H, m), 3.72 (1H, bs), 1.41 (3H, d, J=6.6 Hz). 13C NMR (150 MHz, CDCl₃): δ 147.53, 137.09, 133.31, 129.39, 129.34, 128.65, 127.49, 126.45, 117.46, 113.50, 50.98, 22.25. IR (ν/cm-1): 3412 (br, m), 3081 (w), 3056 (w), 3023 (m), 2968 (m), 2926 (w), 2867 (w), 1602 (s), 1506 (s), 1456 (w), 1429 (w), 1317 (m), 1257 (m), 1178 (m), 1156 (w). LRMS (ES+) [M+H]+ calcd for C₁₆H₁₈N+ 224.14. found: 224.04.

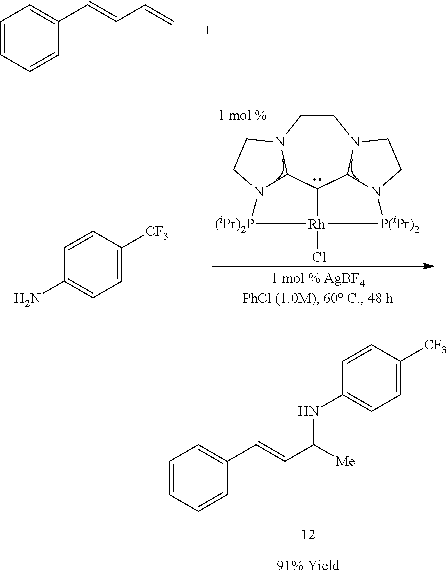

Synthesis of (E)-N-(4-phenylbut-3-en-2-yl)-4-(trifluoromethyl)aniline 12 (Table 2, Entry 2).

Following the general procedure for (CDC)-Rh-catalyzed hydroamination, 4-(trifluoromethyl)aniline (32.2 mg, 0.200 mmol) and phenyl 1,3-butadiene (31.2 mg, 0.240 mmol) were added to a solution of 7 (1.4 mg, 0.0020 mmol) and AgBF₄ (0.4 mg, 0.0020 mmol) in chlorobenzene (200 μL, [ ]=1.00 M) and the reaction allowed to stir at 60° C. for 48 h. The resulting oil was purified by SiO2 column chromatography (10:1 Hex/EtOAc) to afford 12 (53.0 mg, 0.182 mmol, 91% yield) as a colorless oil.

¹H NMR (600 MHz, CDCl₃): δ 7.41 (2H, d, J=8.4 Hz), 7.38 (2H, dd, J=8.2, 1.2 Hz), 7.33 (2H, t, J=7.8 Hz) 7.25 (1H, tt, J=7.2, 1.8 Hz), 6.66 (2H, d, J=8.4 Hz), 6.58 (1H, d, J=6.2 Hz), 6.20 (1H, dd, J=15.9, 5.7 Hz), 4.19-4.22 (1H, m), 4.1 (1H, bs) 1.45 (3H, d, J=6.6 Hz). 13C NMR (150 MHz, CDCl₃): δ 149.75, 131.95, 129.72, 128.56, 127.57, 126.52 (q, J=2.5 Hz), 126.32, 124.97 (q, J=223.8 Hz), 118.68 (q, J=27.5 Hz), 112.38, 50.53, 21.91. 19F NMR (564 MHz, CDCl3): δ 60.89. IR (ν/cm-1): 3418 (br, s), 3083 (w), 3062 (w), 3027 (m), 2973 (m), 2928 (m), 2871 (m), 1616 (s), 1531 (s), 1491 (w), 1327 (s), 1266 (m), 1188 (m), 1159 (m), 1110 (s). LRMS (ES+) [M+H]+ calcd for C₁₇H₁₇NF₃+ 292.13. found: 292.06.

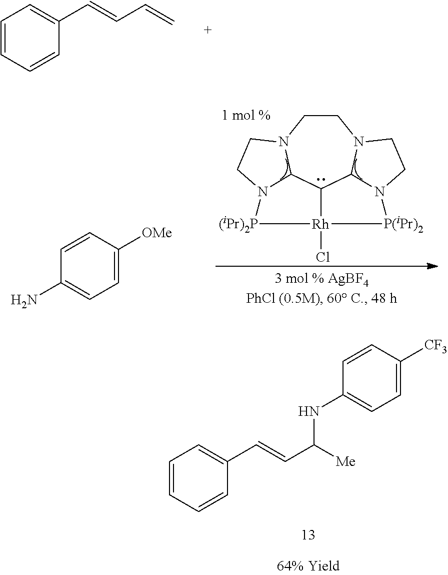

Synthesis of (E)-4-methoxy-N-(4-phenylbut-3-en-2-yl)aniline 13 (Table 2, Entry 3).

Following the general procedure for (CDC)-Rh-catalyzed hydroamination, 4-methoxyaniline (24.6 mg, 0.200 mmol) and phenyl 1,3-butadiene (26.0 mg, 0.200 mmol) were added to a solution of 7 (3.3 mg, 0.0060 mmol) and AgBF₄ (1.2 mg, 0.0062 mmol) in chlorobenzene (400 μL, [ ]=0.500 M), and the reaction allowed to stir at 60° C. for 48 h. The resulting oil was purified by SiO2 column chromatography (10:1 Hex/EtOAc) to afford 13 (32.5 mg, 0.128 mmol, 64% yield) as a colorless oil.

¹H NMR (600 MHz, CDCl3): δ 7.37 (2H, dd, J=8.1, 1.2 Hz), 7.31 (2H, t, J=7.8 Hz), 7.23 (1H, tt, J=7.2, 1.2 Hz), 6.77-6.80 (2H, m), 6.63-6.66 (2H, m), 6.58 (1H, d, J=15.6 Hz), 6.23 (1H, dd, J=16.2, 6.0 Hz), 4.07-4.10 (1H, m), 3.75 (3H, s), 1.40 (3H, d, J=6.6 Hz). 13C NMR (150 MHz, CDCl₃): δ 152.04, 141.56, 136.98, 133.53, 129.19, 128.47, 127.27, 126.26, 114.89, 114.76, 55.72, 51.80, 22.09. IR (ν/cm-1): 3396 (br, m), 3059 (w), 3025 (m), 2964 (m), 2928 (m), 2831 (m), 1502 (s), 1448 (m), 1291 (m), 1234 (s), 1177 (m), 1038 (m). LRMS (ES+) [M+H]+ calcd for C₁₇H₂₀NO+ 254.15. found: 254.05.

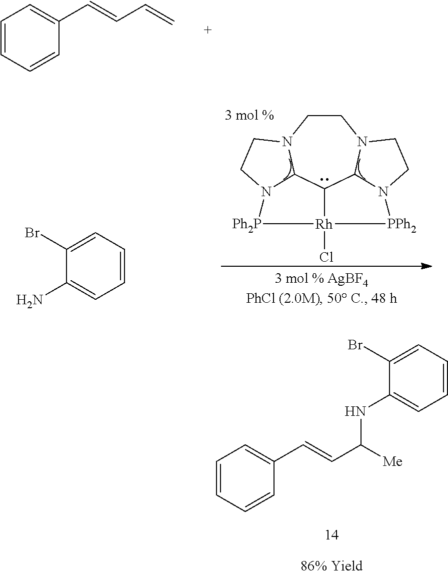

Synthesis of (E)-2-bromo-N-(4-phenylbut-3-en-2-yl)aniline 14 (Table 2, Entry 4).

Following the general procedure for (CDC)-Rh-catalyzed hydroamination, 2-bromoaniline (34.4 mg, 0.200 mmol) and phenyl 1,3-butadiene (39.0 mg, 0.300 mmol) were added to a solution of 6 (4.1 mg, 0.0059 mmol) and AgBF4 (1.2 mg, 0.0062 mmol) in chlorobenzene (100 μL, [ ]=2.00 M), and the reaction allowed to stir at 50° C. for 48 h. The resulting oil was purified by SiO2 column chromatography (15:1 Hex/Et2O) to afford 14 (51.2 mg, 0.172 mmol, 86% yield) as a colorless oil.

¹H NMR (400 MHz, CDCl₃): δ 7.43 (1H, dd, J=7.9, 1.4 Hz), 7.35-7.37 (2H, m), 7.30 (2H, t, J=7.3 Hz), 7.22 (1H, tt, J=6.9, 2.0 Hz), 7.13 (1H, td, J=7.7, 1.3 Hz), 6.69 (1H, dd, J=8.2, 1.1 Hz), 6.53-6.58 (2H, m), 6.21 (1H, dd, J=15.9, 5.9 Hz), 4.41 (1H, bd, J=6.1 Hz), 4.15-4.20 (1H, m), 1.47 (3H, d, J=6.6 Hz). 13C NMR (100 MHz, CDCl₃): δ 144.15, 136.78, 132.47, 132.34, 129.54, 128.53, 128.39, 127.46, 126.36, 117.72, 112.45, 109.72, 50.98, 22.14. IR (ν/cm-1): 3409 (br, m), 3060 (w), 3025 (m), 2967 (m), 2922 (m), 2867 (w), 1595 (s), 1504 (s), 1459 (m), 1426 (m), 1319 (s), 1165 (m), 1018 (m). LRMS (ES+) [M+H]+ calcd for C₁₆H₁₇BrN+ 302.05. found: 302.00.

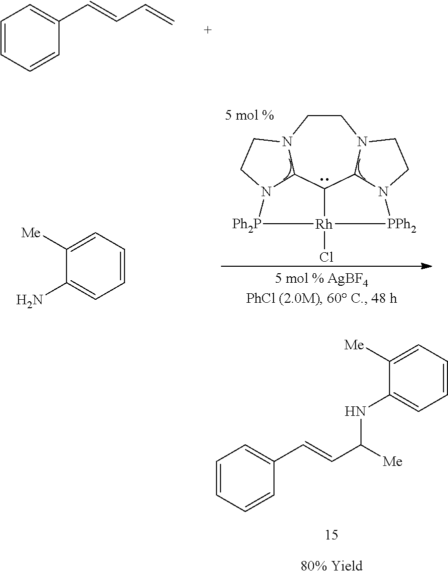

Synthesis of (E)-2-methyl-N-(4-phenylbut-3-en-2-yl)aniline 15 (Table 2, Entry 5).

Following the general procedure for (CDC)-Rh-catalyzed hydroamination, o-toluidine (21.4 mg, 0.200 mmol) and phenyl 1,3-butadiene (39.0 mg, 0.300 mmol) were added to a solution of 7 (5.5 mg, 0.010 mmol) and AgBF₄ (1.9 mg, 0.0098 mmol) in chlorobenzene (100 μL, [ ]=2.00 M), and the reaction allowed to stir at 60° C. for 48 h. The resulting oil was purified by SiO2 column chromatography (15:1 Hex/Et₂O) to afford 15 (38.0 mg, 0.160 mmol, 80% yield) as a colorless oil. ¹H NMR (600 MHz, CDCl₃): δ 7.36 (2H, d, J=7.2 Hz), 7.30 (2H, t, J=7.2 Hz), 7.22 (1H, t, J=7.8 Hz), 6.66 (2H, m), 6.58 (1H, d, J=16.2 Hz), 6.25 (1H, dd, J=16.2, 6.0 Hz), 4.20 (1H, bm), 3.26 (1H, s), 2.19 (3H, s), 1.46 (3H, d, J=7.2 Hz). 13C NMR (150 MHz, CDCl₃): δ 145.47, 137.14, 133.51, 130.23, 129.37, 128.65, 127.48, 127.24, 126.48, 121.84, 116.99, 110.97, 50.85, 22.45, 17.77. IR (ν/cm-1): 3429 (br, m), 3056 (w), 3024 (m), 2967 (m), 2924 (m), 2861 (w), 1605 (s), 1585 (m), 1510 (s), 1477 (w), 1445 (w), 1371 (m), 1314 (m), 1259 (m), 1163 (m), 1050 (m). LRMS (ES+) [M+H]+ calcd for C₁₇H₂₀N+ 238.16. found: 238.13.

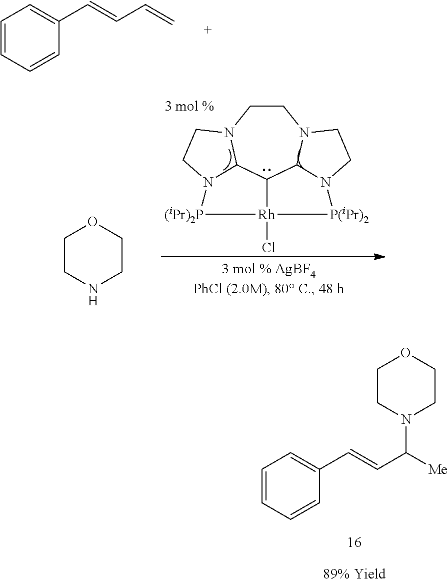

Synthesis of (E)-4-(4-phenylbut-3-en-2-yl)morpholine 16 (Table 2, Entry 6).

Following the general procedure for (CDC)-Rh-catalyzed hydroamination, morpholine (17.4 mg, 0.200 mmol) and phenyl 1,3-butadiene (39.0 mg, 0.300 mmol) were added to a solution of 7 (3.5 mg, 0.0064 mmol) and AgBF4 (1.2 mg, 0.0062 mmol) in chlorobenzene (100 μL, [ ]=2.00 M), and the reaction allowed to stir at 80° C. for 48 h. The resulting oil was purified by SiO2 column chromatography (20:1 CH2Cl₂:MeOH) to afford 16 (38.8 mg, 0.178 mmol, 89% yield) as a yellow oil.

¹H NMR (600 MHz, CDCl₃): δ 7.37 (2H, d, J=7.2 Hz), 7.31 (2H, t, J=7.8 Hz), 7.23 (1H, t, J=7.2 Hz), 6.46 (1H, d, J=16.2 Hz), 6.17 (1H, dd, J=15.9, 8.1 Hz), 3.74 (4H, t, J=6.6 Hz), 3.01-3.04 (1H, m), 2.57 (4H, bt, J=5.1 Hz), 1.26 (3H, d, J=6.6 Hz). 13C NMR (150 MHz, CDCl₃): δ 137.04, 132.27, 131.36, 128.72, 127.61, 126.40, 67.36, 63.27, 50.92, 17.90. IR (ν/cm-1): 3058 (w), 3025 (m), 2961 (s), 2891 (w), 2852 (m), 2806 (m), 2755 (w), 2687 (w), 1494 (m), 1448 (m), 1315 (w), 1266 (m), 1142 (w), 1119 (s), 1069 (w), 1040 (m). LRMS (ES+) [M+H]+ calcd for C₁₄H₂₀NO+ 218.15. found: 218.01.

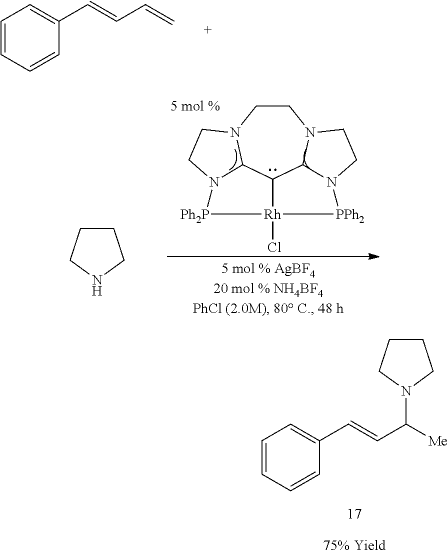

Synthesis of (E)-1-(4-phenylbut-3-en-2-yl)pyrrolidine 17 (Table 2, Entry 7).

Following the general procedure for (CDC)-Rh-catalyzed hydroamination, pyrrolidine (14.2 mg, 0.200 mmol), phenyl 1,3-butadiene (52.1 mg, 0.400 mmol) and NH4BF4 (4.2 mg, 0.040 mmol) were added to a solution of 6 (6.8 mg, 0.0099 mmol) and AgBF4 (1.9 mg, 0.0098 mmol) in chlorobenzene (100 μL, [ ]=2.00 M), and the reaction allowed to stir at 80° C. for 48 h. The resulting oil was purified by SiO2 column chromatography (50:1 CH₂Cl₂/MeOH) to afford 17 (30.2 mg, 0.150 mmol, 75% yield) as a colorless oil.

¹H NMR (600 MHz, CDCl₃): δ 7.37 (2H, d, J=7.3 Hz), 7.29 (2H, t, J=7.7 Hz), 7.21 (1H, t, J=7.3 Hz), 6.47 (1H, d, J=15.8 Hz), 6.24 (1H, dd, J=15.8, 8.5 Hz), 2.90-2.92 (1H, m), 2.56-2.61 (4H, m), 1.77-1.82 (4H, m), 1.3 (3H, d, J=6.5 Hz). 13C NMR (150 MHz, CDCl₃): δ 137.33, 134.07, 129.86, 128.70, 127.44, 126.41, 63.26, 52.40, 23.50, 21.16. IR (ν/cm-1): 3057 (w), 3025 (m), 2968 (s), 2929 (w), 2873 (m), 2781 (s), 1495 (m), 1457 (m), 1370 (w), 1311 (m), 1167 (m), 1139 (m), 1070 (m), 1025 (m). LRMS (ES+) [M+H]+ calcd for C₁₄H₂₀N+ 202.16. found: 202.13.

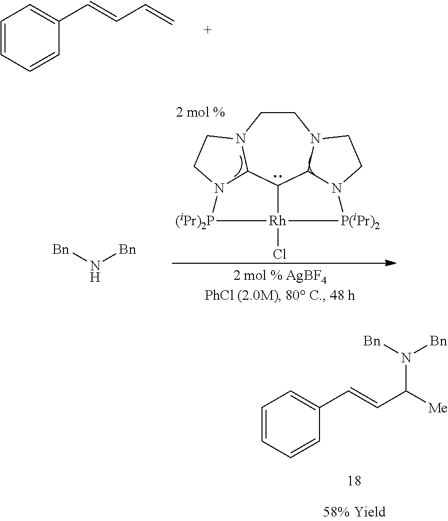

Synthesis of (E)-N,N-dibenzyl-4-phenylbut-3-en-2-amine 18 (Table 2, Entry 8).

Following the general procedure for (CDC)-Rh-catalyzed hydroamination, dibenzylamine (39.4 mg, 0.200 mmol) and phenyl 1,3-butadiene (52.0 mg, 0.400 mmol) were added to a solution of 7 (2.2 mg, 0.0040 mmol) and AgBF4 (0.80 mg, 0.0041 mmol) in chlorobenzene (100 μL, [ ]=2.00 M), and the reaction allowed to stir at 80° C. for 48 h. The resulting oil was purified by SiO₂ column chromatography (5:1 Hex/EtOAc) to afford 18 (38.2 mg, 0.116 mmol, 58% yield) as a colorless oil.

¹H NMR (600 MHz, CDCl₃): δ 7.41-7.44 (6H, m), 7.32-7.36 (6H, m), 6.46 (1H, d, J=16.1 Hz), 6.34 (1H, dd, J=13.5, 6.7 Hz), 3.73 (2H, d, J=14.0 Hz), 3.62 (2H, d, J=13.9 Hz), 3.49-3.52 (1H, m), 1.32 (3H, d, J=6.7 Hz). 13C NMR (150 MHz, CDCl₃): δ 140.58, 137.30, 131.64, 130.93, 128.53, 128.51, 128.17, 127.25, 126.67, 126.25, 54.53, 53.64, 15.80. IR (ν/cm-1): 3061 (w), 3025 (m), 2967 (m), 2928 (m), 2799 (m), 1601 (m), 1494 (m), 1451 (m), 1366 (m), 1144 (m), 1057 (m), 1024 (m). LRMS (ES+) [M+H]+ calcd for C₂₄H₂₆N+ 328.21. found: 328.17.

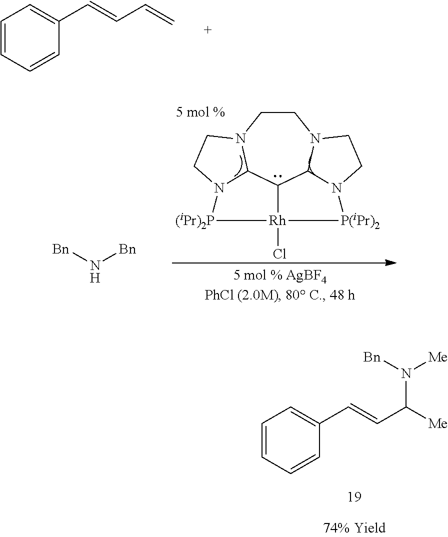

Synthesis of (E)-N-benzyl-N-methyl-4-phenylbut-3-en-2-amine 19 (Table 2, Entry 9).

Following the general procedure for (CDC)-Rh-catalyzed hydroamination, benzylmethylamine (24.2 mg, 0.200 mmol) and phenyl 1,3-butadiene (39.0 mg, 0.300 mmol) were added to a solution of 7 (5.5 mg, 0.010 mmol) and AgBF₄ (1.9 mg, 0.0098 mmol) in chlorobenzene (100 μL, [ ]=2.00 M), and the reaction allowed to stir at 80° C. for 48 h. The resulting oil was purified by SiO2 column chromatography (10:1 Hex/EtOAc to 100% EtOAc) to afford 19 (37.0 mg, 0.148 mmol, 74% yield) as a colorless oil.

¹H NMR (600 MHz, CDCl3): δ 7.42 (2H, d, J=7.2 Hz), 7.32-7.37 (6H, m), 7.23-7.27 (2H, m), 6.49 (1H, d, J=16.2 Hz), 6.33 (1H, dd, J=16.2, 7.2 Hz), 3.67 (1H, d, J=13.2 Hz), 3.53 (1H, d, J=13.2 Hz), 3.37-3.40 (1H, m), 2.24 (3H, s), 1.32 (3H, d, J=6.6 Hz). 13C NMR (150 MHz, CDCl₃): δ 140.06, 137.40, 132.25, 130.89, 129.00, 128.68, 128.35, 127.41, 126.91, 126.40, 60.52, 58.39, 38.06, 17.11. IR (ν/cm-1): 3082 (w), 3060 (w), 3026 (m), 2970 (s), 2932 (w), 2876 (w), 2839 (m), 2788 (s), 1601 (m), 1494 (m), 1450 (s), 1368 (m), 1311 (m), 1209 (w), 1156 (w), 1129 (w), 1073 (m), 1027 (m). LRMS (ES+) [M+H]+ calcd for C₁₈H₂₂N+ 252.17. found: 252.07.

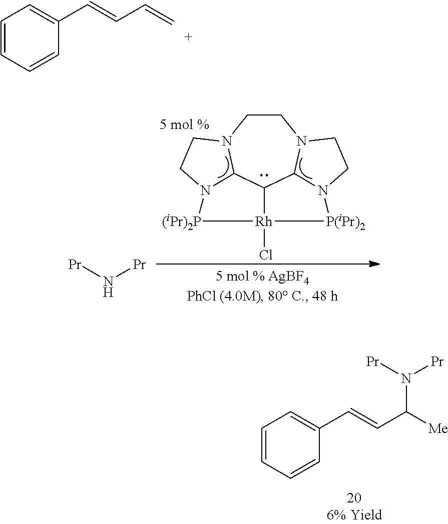

Synthesis of (E)-4-phenyl-N,N-dipropylbut-3-en-2-amine 20 (Table 2, Entry 10).

Following the general procedure for (CDC)-Rh-catalyzed hydroamination, di-n-propyl amine (20.2 mg, 0.200 mmol) and phenyl 1,3-butadiene (39.0 mg, 0.300 mmol) were added to a solution of 7 (5.5 mg, 0.010 mmol) and AgBF₄ (1.9 mg, 0.0098 mmol) in chlorobenzene (50 μL, [ ]=4.00 M), and the reaction allowed to stir at 80° C. for 48 h. The resulting oil was purified by SiO2 column chromatography (100% CH₂Cl₂ to 20:1 CH₂Cl₂:MeOH) to afford 20 (2.7 mg, 0.012 mmol, 6% yield) as a yellow oil.

¹H NMR (400 MHz, CDCl3): δ 7.43 (2H, d, J=9.1 Hz), 7.32-7.37 (3H, m), 6.74 (1H, d, J=15.9 Hz), 6.26 (1H, dd, J=15.9, 8.5 Hz), 4.10-4.17 (1H, m), 3.09-3.11 (4H, m), 1.82 (4H, quintet, J=8.2 Hz), 1.60 (3H, d, J=6.7 Hz), 0.99 (6H, t, J=7.3 Hz). 13C NMR (100 MHz, CDCl3): δ 138.03, 134.83, 129.35, 129.00, 127.18, 121.68, 62.89, 53.04, 18.24, 16.19, 11.2. IR (ν/cm-1): 3140 (br, m), 2972 (m), 2932 (m), 2883 (w), 2852 (w), 1652 (m), 1457 (m), 1061 (s). LRMS (ES+) [M+H]+ calcd for C₁₆H₂₆N+ 232.21. found: 232.18.

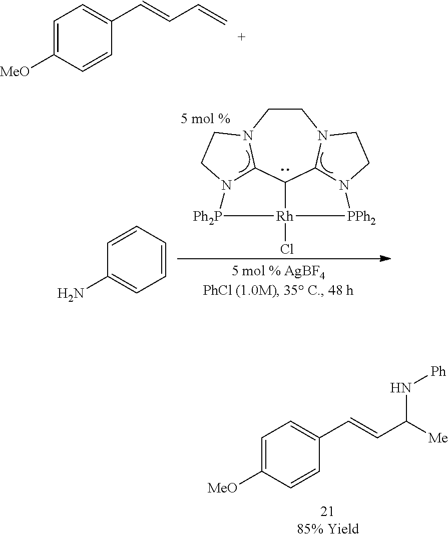

Synthesis of (E)-N-(4-(4-methoxyphenyl)but-3-en-2-yl)aniline 21 (Scheme 2).

Following the general procedure for (CDC)-Rh-catalyzed hydroamination, aniline (18.6 mg, 0.200 mmol) and (E)-1-(buta-1,3-dien-1-yl)-4-methoxybenzene (64.1 mg, 0.400 mmol) were added to a solution of 6 (6.8 mg, 0.0099 mmol) and AgBF4 (1.9 mg, 0.0098 mmol) in chlorobenzene (200 μL, [ ]=1.00 M), and the reaction allowed to stir at 35° C. for 48 h. The resulting oil was purified by SiO₂ column chromatography (20:1 Hex/Et₂O) to afford 21 (43.1 mg, 0.170 mmol, 85% yield) as a light yellow solid.

¹H NMR (600 MHz, CDCl₃): δ 7.30 (2H, d, J=8.7 Hz), 7.17 (2H, t, J=8.0 Hz), 6.85 (2H, d, J=8.7 Hz), 6.70 (1H, t, J=7.3 Hz), 6.66 (2H, d, J=7.6), 6.53 (1H, d, J=15.8), 6.09 (1H, dd, J=15.9, 5.9), 4.13-4.16 (1H, m), 3.81 (3H, s), 3.72 (1H, bs), 1.41 (3H, d, J=6.6 Hz). 13C NMR (150 MHz, CDCl₃): δ 158.98, 147.42, 130.95, 129.72, 129.14, 128.63, 127.41, 117.22, 113.88, 113.35, 55.25, 50.83, 22.12. IR (ν/cm-1): 3407 (br, m), 3052 (w), 3021 (w), 2967 (m), 2922 (m), 2865 (w), 1602 (s), 1507 (s), 1316 (m), 1227 (s), 1157 (m). LRMS (ES+) [M+H]+ calcd for C₁₇H₁₉NO+ 254.15. found: 254.11.

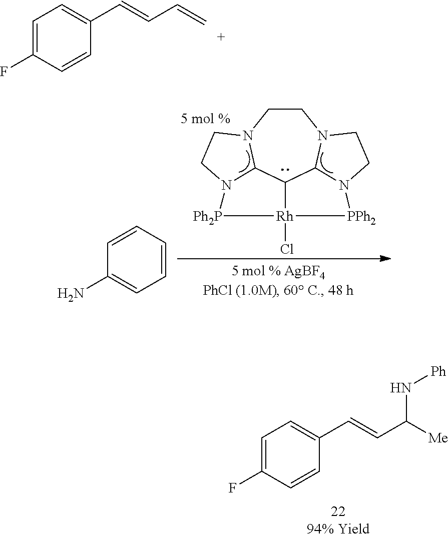

Synthesis of (E)-N-(4-(4-fluorophenyl)but-3-en-2-yl)aniline 22 (Scheme 2).

Following the general procedure for (CDC)-Rh-catalyzed hydroamination, aniline (18.6 mg, 0.200 mmol) and (E)-1-(buta-1,3-diene-1-yl)-4-fluorobenzene (59.3 mg, 0.400 mmol) were added to a solution of 6 (6.8 mg, 0.0099 mmol) and AgBF₄ (1.9 mg, 0.0098 mmol) in chlorobenzene (200 μL, [ ]=1.00 M), and the reaction allowed to stir at 60° C. for 48 h. The resulting oil was purified by SiO2 column chromatography with a layer of 1% (by weight) AgNO3 doped silica gel (20:1 Hex/EtOAc) to afford 22 (45.4 mg, 0.188 mmol, 94% yield) as a light yellow solid.

¹H NMR (600 MHz, CDCl₃): δ 7.31 (2H, m), 7.16 (2H, t, J=7.9 Hz), 6.98 (2H, t, J=8.7 Hz), 6.69 (1H, t, J=7.2 Hz), 6.64 (2H, d, J=7.6 Hz), 6.53 (1H, d, J=15.9 Hz), 6.13 (1H, dd, J=15.9, 5.8 Hz), 4.13 (1H, m, J=6.2 Hz), 3.72 (1H, bs), 1.4 (1H, d, J=6.6 Hz). 13C NMR (150 MHz, CDCl₃): δ 147.35, 133.12, 132.91, 132.89, 129.23, 128.10, 127.82, 127.77, 117.38, 115.47, 115.32, 113.35, 50.79, 22.13. IR (ν/cm-1): 3407 (br, m), 3052 (w), 3021 (w), 2967 (m), 2922 (m), 2865 (w), 1602 (s), 1507 (s), 1316 (m), 1227 (s), 1157 (m). LRMS (ES+) [M+H]+ calcd for C₁₆H₁₇FN+ 242.13. found: 242.14.

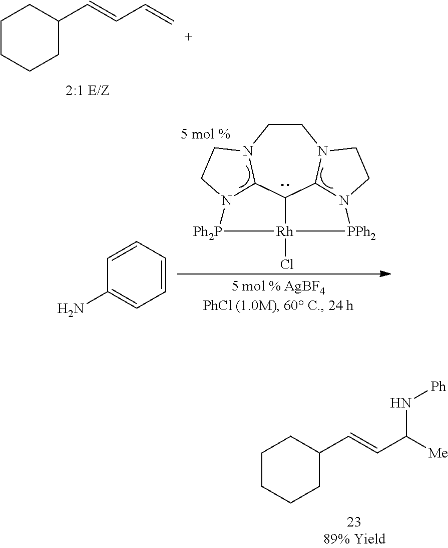

Synthesis of (E)-N-(4-cyclohexylbut-3-en-2-yl)aniline 23 (Table 3, Entry 1).

Following the general procedure for (CDC)-Rh-catalyzed hydroamination, aniline (18.6 mg, 0.200 mmol) and buta-1,3-dien-1-ylcyclohexane (54.5 mg, 0.400 mmol, 2:1 mixture of E/Z isomers) were added to a solution of 6 (6.8 mg, 0.0099 mmol) and AgBF₄ (1.9 mg, 0.0098 mmol) in chlorobenzene (200 μL, [ ]=1.00 M), and the reaction allowed to stir at 60° C. for 24 h. The resulting oil was purified by SiO₂ column chromatography (100% hexanes) to afford an 88:12 mixture of 23 and an unidentifiable constitutional isomer (40.8 mg, 0.178 mmol, 89% combined yield) as a clear oil.

Data is reported for the major product (E)-N-(4-cyclohexylbut-3-en-2-yl)aniline. ¹H NMR (600 MHz, CDCl₃): δ 7.17 (2H, t, J=7.8 Hz), 6.69 (1H, t, J=7.3 Hz), 6.63 (2H, d, J=8.2 Hz), 5.61 (1H, dd, J=15.5, 6.7 Hz), 5.39 (1H, dd, J=15.6, 6.0 Hz), 3.94-3.97 (1H, m), 3.61 (1H, bs), 1.98-1.93 (1H, m), 1.78-1.65 (6H, m), 1.30 (2H, d, J=6.6 Hz), 1.28-1.26 (1H, m), 1.21-1.15 (1H, m), 1.12-1.06 (2H, m). 13C NMR (150 MHz, CDCl3): δ 147.54, 136.43, 130.32, 113.39, 50.59, 40.27, 32.90, 26.04, 22.04. IR (ν/cm-1): 3405 (br, m), 3048 (w), 3017 (w), 2923 (s), 2850 (s), 1601 (s), 1503 (s), 1448 (m), 1318 (m), 1254 (w), 1179 (w). LRMS (ES+) [M+H]+ calcd for C₁₆H₂₄N+ 230.19. found: 230.11.

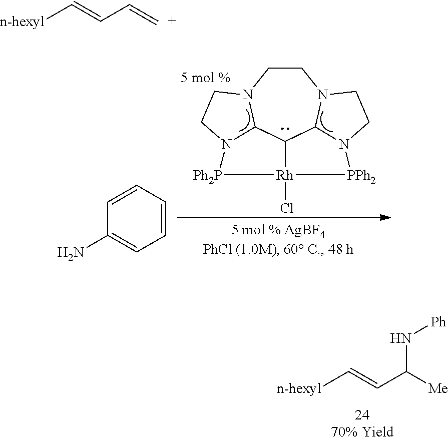

Synthesis of (E)-N-(dec-3-en-2-yl)aniline and (E)-N-(dec-2-en-4-yl)aniline 24 (Table 3, Entry 2).

Following the general procedure for (CDC)-Rh-catalyzed hydroamination, aniline (18.6 mg, 0.200 mmol) and (E)-deca-1,3-diene (55.3 mg, 0.400 mmol) were added to a solution of 6 (6.8 mg, 0.0099 mmol) and AgBF4 (1.9 mg, 0.0098 mmol) in chlorobenzene (200 μL, [ ]=1.00 M), and the reaction allowed to stir at 60° C. for 48 h. The resulting oil was purified by SiO2 column chromatography (20:1 Hex/Et2O) to afford 24 (32.4 mg, 0.140 mmol, 70% combined yield, 3:2 β/δ isomers) as a clear oil.

Reported as a 3:2 mixture of (E)-N-(dec-3-en-2-yl)aniline and (E)-N-(dec-2-en-4-yl)aniline: The regio-isomers were characterized by ¹H COSY NMR (spectra included). 1H NMR (400 MHz, CDCl3): δ [N-(dec-3-en-2-yl)aniline: 7.15 (2H, t, J=7.9 Hz), 6.67 (1H, m), 6.60 (2H, t, J=7.5 Hz), 5.62 (1H, td, J=15.4, 7.0 Hz), 5.41 (1H, dd, J=15.4, 6.0 Hz), 3.90-3.97 (1H, m), 3.59 (1H, bs), 2.02 (2H, q, J=7.1 Hz), 1.27-1.39 (8H, m) 1.29 (3H, d, J=6.6 Hz), 0.88 (3H, t, J=7.1 Hz)], [(E)-N-(dec-2-en-4-yl)aniline: 7.15 (2H, t, J=7.9 Hz), 6.67 (1H, m), 6.60 (2H, t, J=7.5 Hz), 5.62 (1H, td, J=15.4, 7.0 Hz), 5.33 (1H, ddd, J=15.3, 6.6, 1.4 Hz), 3.69-3.75 (1H, m), 3.59 (1H, bs), 1.45-1.64 (2H, m), 1.68 (3H, d, J=6.4 Hz), 1.27-1.39 (8H, m), 0.88 (3H, t, J=6.6 Hz)]. 13C NMR (100 MHz, CDCl₃): δ [Reported as a mixture of N-(dec-3-en-2-yl)aniline and (E)-N-(dec-2-en-4-yl)aniline: 147.76, 147.54, 133.16, 132.91, 130.66, 129.05, 125.89, 117.00, 116.81, 113.38, 113.20, 55.30, 50.51, 36.22, 32.19, 31.68, 29.23, 28.74, 25.94, 22.60, 22.09, 17.70, 14.07]. IR (ν/cm-1): 3410 (br, m), 3053 (w), 3021 (w), 2956 (m), 2926 (s), 2855 (s), 1601 (s), 1504 (s), 1457 (m), 1428 (w), 1374 (w), 1318 (m), 1253 (m), 1179 (w), 1154 (w). LRMS (ES+) [M+H]+ calcd for C₁₆H₂₆N+ 232.21. found: 232.18.

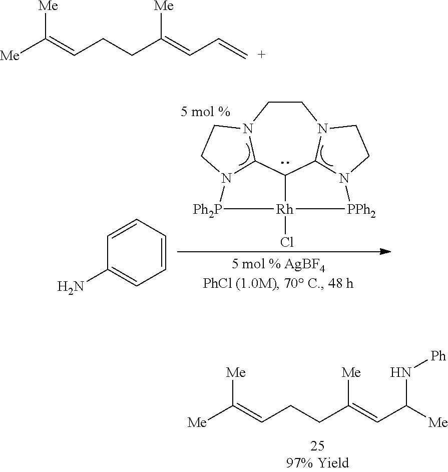

Synthesis of (E)-N-(4,8-dimethylnona-3,7-dien-2-yl)aniline 25 (Table 3, Entry 3).

Following the general procedure for (CDC)-Rh-catalyzed hydroamination, aniline (18.6 mg, 0.200 mmol) and (E)-4,8-dimethylnona-1,3,7-triene (60.1 mg, 0.400 mmol, 92:8 E/Z) were added to a solution of 6 (6.8 mg, 0.0099 mmol) and AgBF4 (1.9 mg, 0.0098 mmol) in chlorobenzene (200 μL, [ ]=1.00 M), and the reaction allowed to stir at 70° C. for 48 h. The resulting oil was purified by SiO2 column chromatography (20:1 Hex/Et₂O) to afford 25 (47.2 mg, 0.194 mmol, 97% yield, 92:8 E/Z) as a clear oil.

Data for the E isomer is reported. ¹H NMR (600 MHz, CDCl₃): δ 7.15 (2H, dt, J=7.0, 1.7 Hz), 6.67 (1H, dt, J=7.3, 0.9 Hz), 6.58 (2H, dd, J=8.5, 0.9 Hz), 5.05-5.07 (2H, m), 4.14 (1H, m), 3.57 (1H, s), 1.98-2.09 (4H, m), 1.73 (3H, d, J=1.2 Hz), 1.66 (3H, d, J=0.8 Hz), 1.59 (3H, s), 1.25 (3H, d, J=6.5 Hz). 13C NMR (150 MHz, CDCl₃): δ 147.79, 136.12, 131.53, 129.59, 129.11, 124.00, 117.03, 113.30, 47.25, 39.43, 26.39, 25.69, 22.01, 17.72, 16.38. IR (ν/cm-1): 3406 (br, m), 3052 (w), 2966 (s), 2924 (s), 2859 (w), 1602 (s), 1502 (s), 1443 (m), 1424 (m), 1378 (m), 1318 (m), 1254 (m), 1150 (m), 1105 (m), 1072 (m). LRMS (ES+) [M+H]+ calcd for C₁₇H₂₆N+ 244.21. found: 244.09.

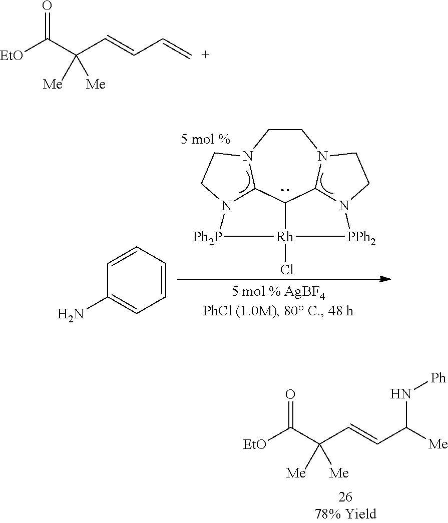

Synthesis of (E)-ethyl 2,2-dimethyl-5-(phenylamino)hex-3-enoate 26 (Table 3, Entry 4).

Following the general procedure for (CDC)-Rh-catalyzed hydroamination, aniline (18.6 mg, 0.200 mmol) and (E)-ethyl-2,2-dimethylhexa-3,5-dienoate (67.3 mg, 0.400 mmol) were added to a solution of 6 (6.8 mg, 0.0099 mmol) and AgBF4 (1.9 mg, 0.0098 mmol) in chlorobenzene (200 μL, [ ]=1.00 M), and the reaction allowed to stir at 80° C. for 48 h. The resulting oil was purified by SiO₂ column chromatography with a layer of 1% (by weight) AgNO3 doped silica (20:1 Hex/Et2O) to afford 26 (40.8 mg, 0.156 mmol, 78% yield) as a clear oil.

¹H NMR (600 MHz, CDCl3): δ 7.13 (2H, t, J=7.8 Hz), 6.67 (1H, t, J=7.2 Hz), 6.59 (2H, d, J=7.7 Hz), 5.83 (1H, dd, J=15.7, 0.5 Hz), 5.47 (1H, dd, J=15.7, 5.9 Hz), 4.08 (2H, q, J=7.1 Hz), 3.95-3.98 (1H, m), 1.29 (3H, d, J=6.5 Hz), 1.27 (6H, s), 1.2 (3H, t, J=7.1 Hz). 13C NMR (150 MHz, CDCl3): δ 176.46, 147.35, 134.73, 131.39, 129.07, 117.31, 113.55, 60.63, 50.76, 43.82, 25.01, 21.99, 14.13. IR (ν/cm-1): 3399 (br, m), 2977 (w), 2933 (s), 2874 (m), 1726 (s), 1603 (s), 1503 (s), 1318 (m), 1254 (m), 1144 (s), 1027 (m). LRMS (ES+) [M+H]+ calcd for C₁₆H₂₄NO₂+ 262.18. found: 262.13.

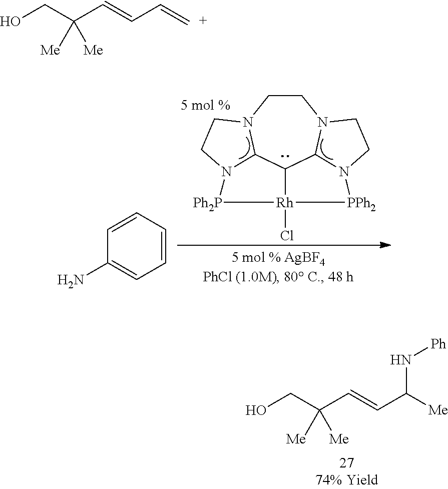

Synthesis of (E)-2,2-dimethyl-5-(phenylamino)hex-3-en-1-ol 27 (Table 3, Entry 5).

Following the general procedure for (CDC)-Rh-catalyzed hydroamination, aniline (18.6 mg, 0.200 mmol) and (E)-2,2-dimethylhexa-3,5-dien-1-ol (50.5 mg, 0.400 mmol) were added to a solution of 6 (6.8 mg, 0.099 mmol) and AgBF₄ (1.9 mg, 0.098 mmol) in chlorobenzene (200 μL, [ ]=1.00 M), and the reaction allowed to stir at 80° C. for 48 h. The resulting oil was purified by SiO2 column chromatography with a layer of 1% (by weight) AgNO3 doped silica (10:1 Hex/Et₂O) to afford 27 (32.5 mg, 0.148 mmol, 74% yield) as a clear oil.

¹H NMR (600 MHz, CDCl3): δ 7.16 (2H, dt, J=7.0, 1.6 Hz), 6.69 (1H, dt, J=7.3, 0.9 Hz), 6.59 (2H, dd, J=8.5, 0.9 Hz), 5.54 (1H, dd, J=15.8, 0.9 Hz), 5.39 (1H, dd, J=15.8, 6.3 Hz), 3.96-3.99 (1H, m), 3.58 (1H, bs), 3.25 (2H, dd, J=14.2, 10.6 Hz), 1.31 (3H, d, J=6.6 Hz), 0.98 (6H, d, J=4.0 Hz). 13C NMR (150 MHz, CDCl₃): δ 147.37, 136.92, 132.10, 129.15, 117.47, 113.73, 71.54, 51.02, 38.29, 23.92, 23.57, 22.21. IR (ν/cm-1): 3360 (br, s), 2959 (s), 2926 (s), 2869 (m), 1743 (s), 1602 (s), 1503 (s), 1461 (m), 1374 (m), 1319 (m), 1254 (m), 1155 (m), 1041 (m). LRMS (ES+) [M+H]+ calcd for C₁₄H₂₂NO+ 220.17. found: 220.11.

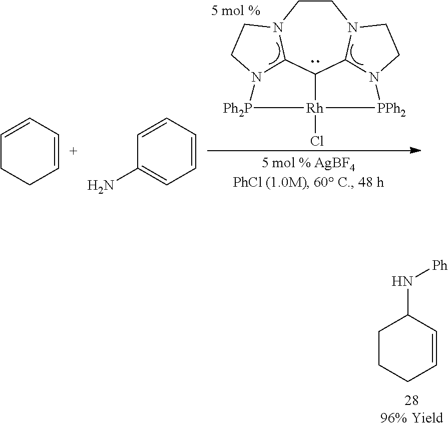

Synthesis of N-(cyclohex-2-en-1-yl)aniline 28 (Table 3, Entry 6).

Following the general procedure for (CDC)-Rh-catalyzed hydroamination, aniline (18.6 mg, 0.200 mmol) and cyclohexa-1,3-diene (64.1 mg, 0.800 mmol) were added to a solution of 6 (6.8 mg, 0.0099 mmol) and AgBF4 (1.9 mg, 0.0098 mmol) in chlorobenzene (200 μL, [ ]=1.00 M), and the reaction allowed to stir at 60° C. for 48 h. The resulting oil was purified by SiO₂ column chromatography (20:1 Hex/Et2O) to afford 28 (33.3 mg, 0.192 mmol, 96% yield) as a clear oil.

¹H NMR (600 MHz, CDCl3): δ 7.17 (2H, dt, J=7.0, 1.6 Hz), 6.69 (1H, t, J=7.1 Hz), 6.62 (2H, d, J=5.0 Hz), 5.84-5.87 (1H, m), 5.75-5.77 (1H, m), 4.00 (1H, bs), 3.63 (1H, bs), 1.99-2.09 (2H, m), 1.89-1.93 (1H, m), 1.69-1.75 (1H, m), 1.60-1.67 (2H, m). 13C NMR (150 MHz, CDCl₃): δ 147.20, 130.15, 129.33, 128.59, 117.14, 113.23, 47.86, 28.90, 25.18, 19.67. IR (ν/cm-1): 3404 (br, m), 3083 (w), 3050 (w), 3021 (m), 2925 (s), 2859 (m), 2360 (w), 1603 (s), 1558 (s), 1504 (m), 1429 (m), 1309 (m), 1257 (m), 1179 (w), 1102 (m). LRMS (ES+) [M+H]+ calcd for C₁₂H₁₆N+ 174.13. found: 174.05.

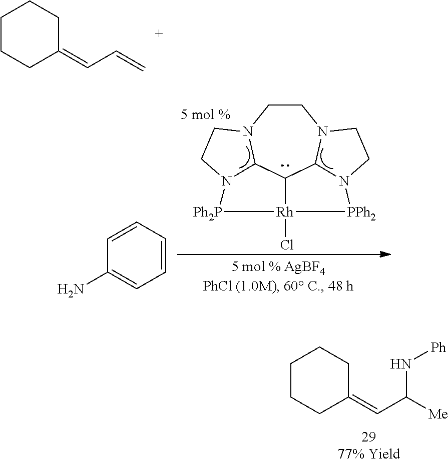

Synthesis of N-(1-cyclohexylidenepropan-2-yl)aniline 29 (Table 3, Entry 7).

Following the general procedure for (CDC)-Rh-catalyzed hydroamination, aniline (18.6 mg, 0.200 mmol) and allylidenecylohexane (48.9 mg, 0.400 mmol) were added to a solution of 6 (6.8 mg, 0.0099 mmol) and AgBF4 (1.9 mg, 0.0098 mmol) in chlorobenzene (200 μL, [ ]=1.00 M), and the reaction allowed to stir at 60° C. for 48 h. The resulting oil was purified by SiO2 column chromatography (20:1 Hex/Et₂O) to afford 29 (33.2 mg, 0.154 mmol, 77% yield) as colorless oil.

¹H NMR (400 MHz, CDCl3): δ 7.15 (2H, dt, J=8.0, 2.1 Hz), 6.66 (2H, t, J=7.3 Hz), 6.59 (2H, dd, J=8.6, 1.0 Hz), 4.99 (3H, d, J=8.4 Hz), 4.17-4.24 (1H, m), 3.57 (1H, bs), 2.20-2.28 (2H, m), 2.04-2.07 (2H, m), 1.48-1.61 (6H, bm), 1.25 (3H, d, J=6.5 Hz). 13C NMR (100 MHz, CDCl₃): δ 140.68, 129.09, 126.20, 117.02, 113.36, 46.36, 36.92, 29.27, 28.51, 27.78, 26.78, 22.60. IR (ν/cm-1): 3405 (br, m), 3083 (w), 3050 (w), 3018 (w), 2926 (s), 2852 (s), 1666 (w), 1601 (s), 1503 (s), 1447 (m), 1374 (w), 1317 (m), 1253 (w), 1179 (w), 1154 (w), 1111 (w), 1074 (w), 1029 (w). LRMS (ES+) [M+K]+ calcd for C₁₅H₂₁NK+ 254.13. found: 254.03.

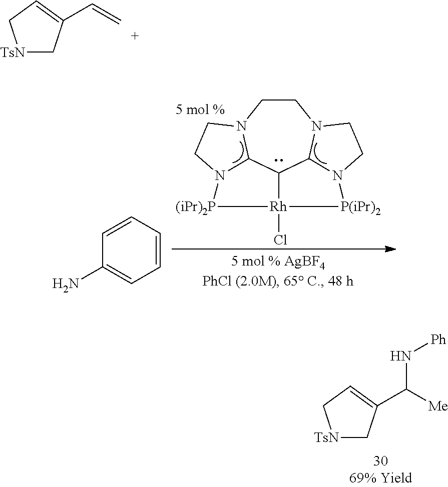

Synthesis of N-(1-(1-tosyl-2,5-dihydro-1H-pyrrol-3-yl)ethyl)aniline 30 (Table 3, Entry 8).

Following the general procedure for (CDC)-Rh-catalyzed hydroamination, aniline (18.6 mg, 0.200 mmol) and 1-tosyl-3-vinyl-2,5-dihydro-1H-pyrrole (99.7 mg, 0.400 mmol) were added to a solution of 7 (5.5 mg, 0.010 mmol) and AgBF4 (1.9 mg, 0.0098 mmol) in chlorobenzene (100 μL, [ ]=2.00 M), and the reaction allowed to stir at 65° C. for 48 h. The resulting oil was purified by SiO₂ column chromatography (20:1 to 100% Et2O) to afford 30 (47.1 mg, 0.138 mmol, 69% yield) as a light yellow oil.

¹H NMR (600 MHz, CDCl₃): δ 7.66 (2H, d, J=8.2 Hz), 7.27 (2H, d, J=8.0 Hz), 7.09 (2H, t, J=7.9 Hz), 6.69 (1H, t, J=7.3 Hz), 6.45 (2H, d, J=7.9 Hz), 5.48-5.49 (1H, m), 4.03-4.18 (4H, m), 4.00 (1H, m), 3.52 (1H, bs), 2.43 (3H, m), 1.29 (2H, d, J=6.7 Hz). 13C NMR (150 MHz, CDCl₃): δ 146.72, 143.34, 142.50, 134.09, 129.71, 129.19, 127.33, 119.30, 117.73, 113.06, 55.04, 54.32, 47.84, 21.51, 20.57. IR (ν/cm-1): 3391 (br, m), 3052 (w), 3019 (w), 2962 (w), 2923 (m), 2859 (m), 1602 (s), 1506 (s), 1338 (s), 1254 (m), 1162 (s), 1099 (m). LRMS (ES+) [M+H]+ calcd for C₁₉H₂₃N₂O₂S+ 343.15. found: 343.14.

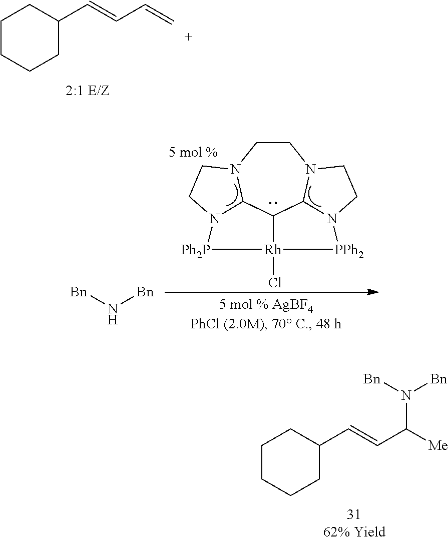

Synthesis of (E)-N,N-dibenzyl-4-cyclohexylbut-3-en-2-amine 31 (Table 4, Entry 1).

Following the general procedure for (CDC)-Rh-catalyzed hydroamination, dibenzylamine (39.5 mg, 0.200 mmol) and buta-1,3-dien-1-ylcyclohexane (54.5 mg, 0.400 mmol, 2:1 E/Z) were added to a solution of 6 (6.8 mg, 0.0099 mmol) and AgBF4 (1.9 mg, 0.0098 mmol) in chlorobenzene (100 μL, [ ]=2.00 M), and the reaction allowed to stir at 70° C. for 48 h. The resulting oil was purified by SiO₂ column chromatography with a layer of 1% (by weight) AgNO₃ doped silica (20:1 Hex/Et₂O) to afford 31 (41.4 mg, 0.124 mmol, 62% yield) as a colorless oil.

¹H NMR (600 MHz, CDCl₃): δ 7.38 (4H, d, J=7.4 Hz), 7.28 (4H, t, J=7.5 Hz), 7.20 (2H, t, J=7.2 Hz), 5.39-5.47 (2H, m), 3.62 (2H, d, J=13.9 Hz), 3.48 (2H, d, J=13.9 Hz), 3.21-3.24 (1H, m), 1.95-2.00 (1H, m), 1.66-1.75 (4H, m), 1.64-1.66 (1H, m), 1.25-1.33 (3H, m), 1.15 (2H, d, J=6.7 Hz), 1.05-1.18 (2H, m). 13C NMR (150 MHz, CDCl3): δ 140.94, 138.41, 128.57, 128.09, 127.99, 126.54, 54.50, 53.45, 40.78, 33.39, 33.31, 26.23, 26.10, 16.16. IR (ν/cm-1): 3063 (w), 3026 (m), 2963 (w), 2923 (s), 2850 (m), 2796 (w), 1494 (w), 1450 (m), 1376 (m), 1147 (w), 1073 (w), 1028 (w). LRMS (ES+) [M+H]+ calcd for C₂₄H₃₂N+ 334.25. found: 334.21.

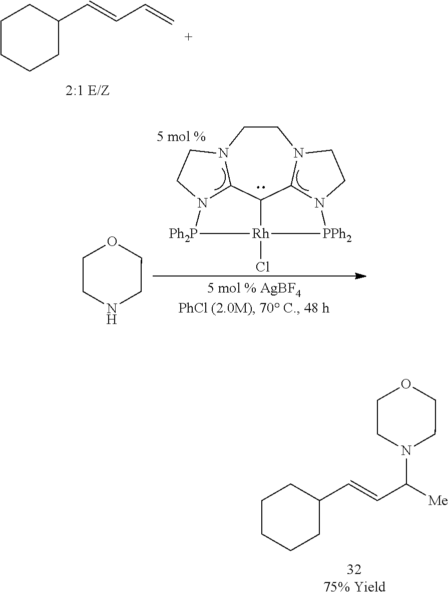

Synthesis of (E)-4-(4-cyclohexylbut-3-en-2-yl)morpholine 32 (Table 4, Entry 2).

Following the general procedure for (CDC)-Rh-catalyzed hydroamination, morpholine (17.4 mg, 0.200 mmol) and buta-1,3-dien-1-ylcyclohexane (54.5 mg, 0.400 mmol, 2:1 E/Z) were added to a solution of 6 (6.8 mg, 0.0099 mmol) and AgBF₄ (1.9 mg, 0.0098 mmol) in chlorobenzene (100 μL, [ ]=2.00 M), and the reaction allowed to stir at 90° C. for 48 h. The resulting oil was purified by SiO2 column chromatography (3:1 Hex/Et2O) to afford a 89:11 mixture of 32 and an unidentifiable constitutional isomer (33.5 mg, 0.150 mmol, 75% combined yield) as a clear oil. Data is reported for the major product (E)-4-(4-cyclohexylbut-3-en-2-yl)morpholine.

¹H NMR (600 MHz, CDCl₃): δ 5.45 (1H, dd, J=15.5, 6.5 Hz), 5.28 (1H, ddd, J=15.5, 8.2, 1.2 Hz), 3.69-3.71 (4H, m), 2.74-2.78 (1H, m), 2.41-2.53 (4H, m), 1.90-1.95 (1H, m), 1.67-1.71 (4H, m), 1.60-1.65 (1H, m), 1.23-1.30 (2H, m), 1.13-1.18 (1H, m), 1.13 (3H, d, J=6.5 Hz), 1.01-1.09 (2H, m). 13C NMR (150 MHz, CDCl3): δ 138.52, 128.92, 67.22, 62.89, 50.52, 40.41, 33.04, 32.97, 26.14, 25.98, 18.06. IR (ν/cm-1): 2958 (w), 2924 (s), 2851 (s), 2802 (m), 1448 (m), 1265 (m) 1245 (w), 1198 (s). LRMS (ES+) [M+H]+ calcd for C₂₄H₃₂N+ 224.20. found: 224.16.

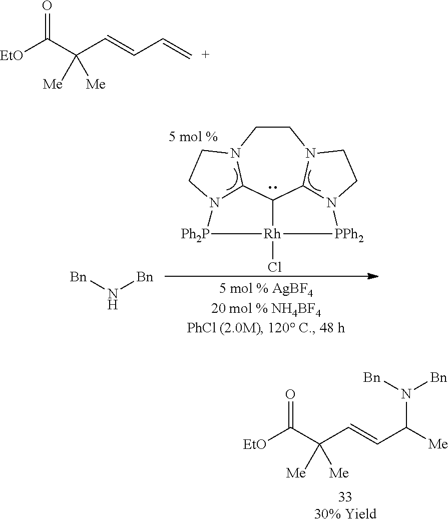

Synthesis of (E)-ethyl 5-(dibenzylamino)-2,2-dimethylhex-3-enoate 33 (Table 4, Entry 3).

Following the general procedure for (CDC)-Rh-catalyzed hydroamination, dibenzylamine (19.7 mg, 0.100 mmol), (E)-ethyl 2,2-dimethylhexa-3,5-dienoate (33.6 mg, 0.200 mmol) and NH₄BF₄ (2.1 mg, 0.02 mmol) were added to a solution of 6 (3.4 mg, 0.0050 mmol) and AgBF₄ (1.0 mg, 0.0051 mmol) in chlorobenzene (50 μL, [ ]=2.0 M), and the reaction allowed to stir at 120° C. for 48 h. The resulting oil was purified by SiO₂ column chromatography (100:1 Hex/Et₂O) to afford 33 (11.0 mg, 0.030 mmol, 30% yield) as a colorless oil.

¹H NMR (600 MHz, CDCl₃): δ 7.37 (4H, d, J=7.1 Hz), 7.30 (4H, t, J=3.6 Hz), 7.20 (2H, t, J=7.2 Hz), 5.69 (1H, dd, J=15.9, 1.0 Hz), 5.56 (1H, dd, J=15.9, 6.7 Hz), 4.12 (2H, q, J=7.1 Hz), 3.63 (1H, d, J=13.9 Hz), 3.47 (1H, d, J=13.9 Hz), 3.25-3.35 (1H, m), 1.30 (6H, d, J=16.4 Hz), 1.22 (3H, t, J=7.1 Hz), 1.17 (3H, d, J=6.8 Hz). 13C NMR (150 MHz, CDCl3): δ 176.68, 140.66, 136.39, 129.29, 128.56, 128.14, 126.65, 60.64, 54.40, 53.50, 44.11, 25.38, 25.10, 15.88, 14.20. IR (ν/cm-1): 3061 (w), 3027 (w), 2965 (m), 2927 (m), 2866 (w), 2801 (w), 1731 (s), 1495 (w), 1455 (m), 1363 (m), 1249 (m), 1143 (s), 1029 (m). LRMS (ES+) [M+H]+ calcd for C₂₄H₃₁NO₂+ 366.24. found: 366.22.

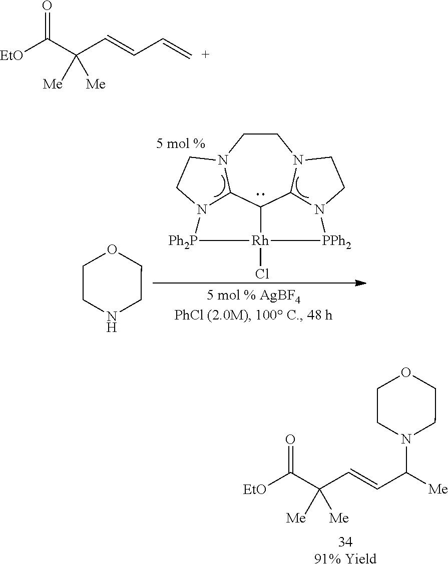

Synthesis of (E)-ethyl 2,2-dimethyl-5-morpholinohex-3-enoate 34 (Table 4, Entry 4).

Following the general procedure for (CDC)-Rh-catalyzed hydroamination, morpholine (17.0 mg, 0.200 mmol) and (E)-ethyl 2,2-dimethylhexa-3,5-dienoate (67.3 mg, 0.400 mmol) were added to a solution of 6 (6.8 mg, 0.0099 mmol) and AgBF₄ (1.9 mg, 0.0098 mmol) in chlorobenzene (100 μL, [ ]=2.00 M), and the reaction allowed to stir at 100° C. for 48 h. The resulting oil was purified by SiO2 column chromatography (10:1 Hex/Et₂O) to afford 34 (46.5 mg, 0.182 mmol, 91% yield) as a clear oil.

¹H NMR (600 MHz, CDCl3): δ 5.73 (1H, dd, J=15.7, 0.5 Hz), 5.43 (1H, dd, J=15.8, 8.3 Hz), 4.11 (2H, q, J=6.1 Hz), 3.71 (4H, t, J=4.7 Hz), 2.82-2.85 (1H, m), 2.45-2.52 (4H, m), 1.29 (6H, d, J=5.8 Hz), 1.23 (3H, t, J=7.1 Hz), 1.15 (3H, d, J=6.5 Hz). 13C NMR (125 MHz, CDCl₃): δ 176.46, 136.59, 130.30, 67.23, 62.71, 60.63, 50.60, 44.04, 25.11, 17.90, 14.15. IR (ν/cm-1): 2975 (s), 2852 (m), 2805 (m), 1731 (s), 1558 (w), 1541 (w), 1507 (w), 1457 (m), 1226 (br, m), 1144 (s), 1119 (m), 1029 (m). LRMS (ES+) [M+H]+ calcd for C₁₄H₂₆NO₃+ 256.19. found: 256.07.

### SUPPORTING INFORMATION REFERENCES

(1) Lishchynskyi, A.; Muniz, K. Chem. Eur. J. 2012, 18, 2212-2216.
(2) Preuβ, T.; Saak, W.; Doye, S. Chem. Eur. J. 2013, 19, 3833-3837.
(3) Quinn, M.; Yao, M.; Yong, L.; Kabalka, G. Synthesis, 2011, 23, 3815-3820.
(4) Townsend, M.; Schrock, R.; Hoveyda, A. J. Am. Chem. Soc. 2012, 134, 11334-11337.
(5) Leopold, E. Org. Synth. 1986, 64, 164-171.
(6) Bigley, D.; Weatherhead, R. J. Chem. Soc., Perkin Trans. 2 1976, 704-706.
(7) Snider, B.; Phillips, G.; Cordova, R. J. Org. Chem. 1983, 48, 3003-3010.
(8) Maier, M. et. al. J. Org. Chem. 2002, 67, 2474-2480.
(9) Kliman, L.; Mlynarski, S.; Ferris, G.; Morken, J. Angew. Chem. Int. Ed. 2012, 51, 521-524.
(10) Lawrence, B. J. Chromatogr. 1968, 38, 535-537.
(11) Schwesinger, R., Angew. Chem. Int. Ed. Engl. 1987, 26, 1164-1165.

The foregoing is illustrative of the present invention, and is not to be construed as limiting thereof. The invention is defined by the following claims, with equivalents of the claims to be included therein.

1. An organometallic complex, comprising:
(a) a tridentate bis(phosphine)-carbodicarbene ligand, and
(b) a transition metal.

2. The complex of claim 1, wherein said ligand has the structure of Formula I: 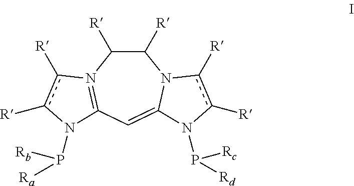
wherein:
each dashed line independently represents an optional double bond;
Rₐ, R_{b}, R_{c}, and R_{d} are each independently selected alkyl, aryl, arylalkyl, alkoxy, amino, or substituted amino;
each R′ is an independently selected hydrogen, hydrocarbyl group, electron donating group, or electron-withdrawing group;
or at least one R′ is S-L-, where S is a solid support and L is a linking group.

3. The complex of claim 2, wherein said ligand has the structure of Formula Ia, Formula Ib, or Formula Ic:

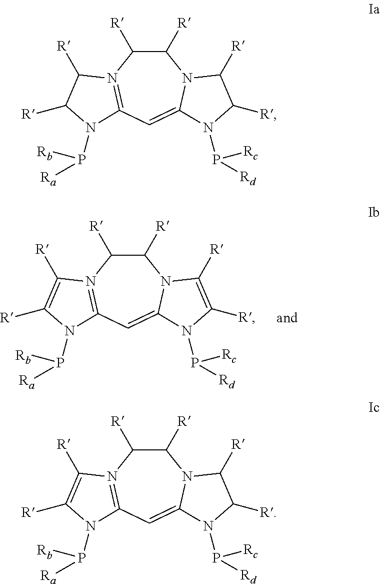

4. The complex of claim 1, wherein said transition metal is selected from the group consisting of ruthenium, nickel, palladium, platinum, rhodium, iridium, cobalt, iron, silver, gold, and molybdenum.

5. The complex of claim 2, wherein Rₐ, R_{b}, R_{c}, and R_{d} are each independently selected alkyl or aryl.

6. The complex of claim 2, wherein at least one R′ is S-L-, where S is a solid support and L is a linking group.

7. The complex of claim 2, wherein each R′ is independently hydrogen, halo, loweralkyl, loweralkoxy, or hydroxyl.

8. A reaction mixture comprising an organometallic complex of claim 1, a solvent, a 1-3, diene substrate, and a substituted amine substrate.

9. The reaction mixture of claim 8, wherein said 1,3-diene has the structure of Formula III: 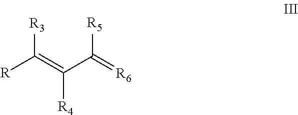
wherein:
R is hydrocarbyl;
R₃, R₄ and R₅ are independently selected H or hydrocarbyl; and
R₆ is alkyl or arylalkyl;
or a pair of R₃, R₄, and R₅ optionally form a linking group.

10. The reaction mixture of claim 8, wherein said substituted amine has the structure of Formula IV: 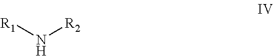
wherein R₁ and R₂ are independently selected hydrocarbyl groups.

11. A method of making an allylic amine, comprising:
reacting a 1,3-diene with a substituted amine in the presence of an organometallic complex of claim 1 in a catalytic amount to produce by intermolecular hydroamination said allylic amine.

12. The method of claim 11, wherein said allylic amine has the structure of Formula II: 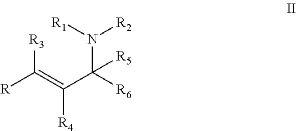
wherein:
R, R₁, and R₂ are independently selected hydrocarbyl groups;
R₃, R₄ and R₅ are independently selected hydrogen or hydrocarbyl groups; and
R₆ is alkyl (e.g., methyl) or arylalkyl (e.g., benzyl).

13. The method of claim 11, wherein said 1,3-diene has the structure of Formula III: 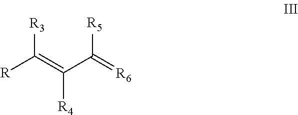
wherein:
R is hydrocarbyl;
R₃, R₄ and R₅ are independently selected H or hydrocarbyl; and
R₆ is alkyl or arylalkyl;
or a pair of R₃, R₄, and R₅ optionally form a linking group.

14. A tridentate bis(phosphine)-carbodicarbene pincer ligand.

15. The ligand of claim 14, having the structure of Formula I: 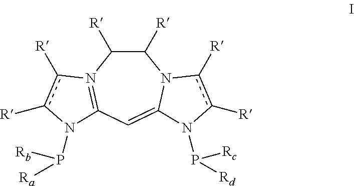
wherein:
each dashed line independently represents an optional double bond;
Rₐ, R_{b}, R_{c}, and R_{d} are each independently selected alkyl, aryl, arylalkyl, alkoxy, amino, or substituted amino;
each R′ is an independently selected hydrogen, hydrocarbyl group, electron donating group, or electron-withdrawing group;
or at least one R′ is S-L-, where S is a solid support and L is a linking group.
